(19) United States
(12) Patent Application Publication
(10) Pub. No.: US 2010/0305198 A1
(43) Pub. Date: Dec. 2, 2010

(54) CATIONIC LIPIDS

(75) Inventors: Mark Bradley, Edinburgh (GB); Asier Unciti-Broceta, Edinburgh (GB)

Correspondence Address: FOLEY AND LARDNER LLP;SUITE 500, 3000 K STREET NW, WASHINGTON, DC, 20007, US

(73) Assignee: THE UNIVERSITY COURT OF THE UNIVERSITY OF EDINBURG, Edinburgh (GB)

(21) Appl. No.: 12/738,628

(22) PCT Filed: Oct. 17, 2008
(86) PCT No.: PCT/GB2008/003527

§ 371 Date: Aug. 11, 2010

(30) Foreign Application Priority Data
Oct. 19, 2007 (GB) 0720486.0

(51) Int. Cl. A61K 48/00; C07C 229/04; C12N 5/07; C12N 15/09; A61P 31/12

(52) U.S. Cl. 514/44 R; 560/171; 435/404; 435/455

(57) ABSTRACT

This invention relates to cationic lipids. More particularly the invention relates to biodegradable cationic lipids having a plurality of cationic headgroups and one or more lipophilic tail groups. The lipids are of utility in various applications, and in particular in permitting transfection of molecules, and in particular DNA and RNA, into cells. As such the lipids have specific utility in the field of gene therapy as well as other applications such as delivery of small molecules into cells, detergents, and metal ion complexation for medical or industrial applications.

### FIELD OF THE INVENTION

This invention relates to cationic lipids. More particularly the invention relates to biodegradable cationic lipids having a plurality of cationic headgroups and one or more lipophilic tail groups. The lipids are of utility in various applications, and in particular in permitting transfection of molecules, and in particular DNA and RNA, into cells. As such the lipids have specific utility in the field of gene therapy as well as other applications such as delivery of small molecules into cells, detergents, and metal ion complexation for medical or industrial applications.

### BACKGROUND OF THE INVENTION

Viral gene delivery, as a molecular biology tool or as a potential therapy, is without doubt the most efficient method of DNA delivery, or transfection, found to date. However, although viral vectors are generally more efficient than non-viral vectors, due to the disadvantages of viral vectors, such as antigenicity, production cost, limited size of cargo, etc, non-viral delivery systems represent a very attractive alternative, especially because of their relatively low cost and procedural simplicity. Despite numerous improvements, however, the in vivo efficacy of non-viral vectors still needs to be increased for both clinical and research purposes, which have been most widely studied to date.

Among the many non-viral vectors, cationic lipids are perhaps the class of compounds that have been most widely studied to date. For a detailed review see B. Martin et al., Curr. Pharm. Design, 2005, 11, 375-394.

As is well-known and well-understood in the art cationic lipids comprise three main parts: a lipophilic component attached through a linking moiety to a positively charged, polar headgroup. The positively charged, polar headgroup is typically the result of protonation of one or more amino groups, or may arise by the provision of a quarternary amine, which bears a permanent positive charge.

When cationic lipids are mixed with DNA or RNA, or other molecules, in an aqueous solution, electrostatic and hydrophobic interactions are known to lead to self-assembly and self-organization via a multi-step mechanism into a liposome-like complex known as a lipoplex. At the end of this multi-step process, the DNA or RNA is condensed, generally the cationic lipids totally envelop the plasmid (providing shielding from nucleases in the surrounding environment) and the surface of the complex has a smooth appearance, which implies the DNA or RNA is properly packaged. Use of an excess of cationic lipid provides the surface with a positive charge, which is postulated to mediate cellular uptake (via non-specific endocytosis) following an interaction with negatively charged cell surface structures such as phospholipids, or heparin sulphates or other proteoglycans.

The first reported cationic lipid, DOTMA, (N-(1-(2,3-dioleyloxy)propyl)-N,N,N-trimethyl-ammonium chloride), was reported in 1987. Now, an enormous range of cationic lipids have been synthesized (see B Martin et al., infra) and several are commercially available. These include Lipofectamine™ 2000 (Invitrogen) and Effectene® Transfection Reagent (Qiagen).

Lipoplex formulation is often assisted by the addition of a neutral surfactant, such as dioleyl phosphatidyl ethanolamine (DOPE), which is believed to improve the transfection abilities of the mixture (Farhood, H. et al., Biochim Biophys Acta, 1995, 1235, 289-295). Due to its fusogenic properties (Farhood, et al., (infra); Ellens, H. et al, Biochemistry, 1986, 25, 4141-7; and Koltover, I. et al., Science, 1998, 281, 78-81) this so-called “co-lipid” appears to drive lipoplex assembly (promotes transition from lamellar to hexagonal phase) by increasing the release of counterions, although DOPE itself is not required for lipoplex assembly. The presence of DOPE is thought to loosen the binding of the cationic lipid to the DNA and enhance endosomal escape of the lipoplexes in to the cytoplasm, a step which is probably the most important in the entire transfection process.

Typically, the headgroups found in cationic lipids are nitrogen-based, since these are protonated at physiological pH and the resultant positive charge assists in the binding of the polyanionic backbone of DNA and RNA. Generally the lipid moiety is composed of two long chain fatty acids or a cholesterol-based derivative. Other than with cholesterol-based cationic lipids the hydrophobic moiety of cationic lipids generally contains unsaturated or saturated alkyl or acyl chains, with a chain length of 12-18 carbon atoms. Long saturated tails tend to display relatively strong intermolecular interactions and a low propensity for hydration and mixing with neutral helper lipids such as DOPE. Furthermore, the addition of double bonds leads to less compact crystal packing. Hence, hydrocarbon tail length and saturation affect lipoplex intradynamics and ultimately the packing efficiency of DNA. Although the majority of cationic lipids have two chains, there is on-going research focusing on the use of single chain detergents that are capable of dimerisation via oxidation. In addition, cholesterol-based tails have been used as an alternative to aliphatic chains since cholesterol is rigid and biodegradable, although cholesterol is also used as an alternative co-lipid to DOPE. Although single-chained agents may be expected to complex DNA by forming micelles, such vectors are often considered to be more toxic and less efficient than their double-tailed counterparts (see Lv, H. et al., J. Control. Release, 2006, 114, 100-109). In particular single-tailed surfactants are less prone to form liposomes (see Remy, J.-S. et al., Bioconjug. Chem., 1994, 5, 647-54; Jakubowski, H., Biochemistry Online, Ch 1, E). However, this is not universally believed to be correct (see Tang, F. X., Hughes, J. A., J. Control. Release, 1999, 62, 345-358; and Yingyongnarongkul, B. E. et al., Chem.-Eur. J, 2004, 10, 463-473.

As the packing properties of cationic lipids are important for optimal condensation of DNA (see Bloomfield, V. A., Curr. Opin. Struct. Biol., 1996, 6, 334-341) the structures of cationic lipids are generally very carefully designed to enable effective DNA-binding and so lipoplex formation. Indeed the prior art is replete with reports of studies into the rational design of cationic lipids by way of modification of the three main parts (i.e. the lipophilic component, the linking motif and the headgroup), which studies have allowed the elucidation of structure-activity relationships. For example it is postulated that the larger the imbalance between the cross-sectional area of the cationic (small) end and the large hydrophobic moiety, the more ‘cone-shaped’ the cationic lipid, which is believed by some to create greater instability in the resulting lipid assembly. Such instability can lead to improved transfection with DNA or RNA release in to the cytoplasm improved.

Hydrophobic and hydrophilic portions of cationic lipids have generally been joined using amide, ether, ester or carbamate bonds, although there is no optimal bond. Ether bonds are quite stable but more toxic than ester bonds, with carbamate viewed as a good balance between stability and toxicity. The linking bond may be considered to determine the cationic amphiphilic lipid's stability, thereby controlling the balance between persistence and toxicity, which are probably related to the half-life in the cells. In terms of linker design there are a great number of reports including the use of photosensitive bonds and the incorporation of environmental sensitive groups, where intracellular hydrolysis leads to controlled DNA delivery at defined stages during intracellular lipoplex trafficking.

Notwithstanding all the prior art, however, there is still a requirement for alternative cationic lipids that offer one or more properties of reduced cell toxicity, promotion of endosomal escape of molecules, e.g. nucleic acids, and, where the molecule to be transfected is a DNA molecule, folding of that DNA.

Despite the vast amount of work undertaken to date in the field of cationic lipids, therefore, it is nevertheless desired to develop further cationic lipids capable of ameliorating or obviating one or more of the problems of in vivo efficacy of the transfection process, toxicity, cost and simplicity of design.

### BRIEF SUMMARY OF THE INVENTION

We have surprisingly found that cationic lipids that comprise pluralities of cationic moieties, or precursors to cationic moieties, connected via ester bonds to a linker moiety to which linker moiety is attached a lipophilic domain through a nitrogen-containing bond show excellent transfection ability. Moreover such cationic lipids are relatively non-toxic since the cationic headgroups, that is to say groups within which the cationic moieties or precursors are contained, may be provided (as is discussed in greater detail below) by natural metabolites or amino acids such as glycine, β-alanine or GABA; the lipophilic tail is typically a cholesterol derivative or a fatty acid, e.g. one or two fatty acid tails; and the cationic lipids can be efficiently broken down by virtue of the presence of the ester and nitrogen-containing bonds to these relatively non-toxic constituent parts.

Viewed from one aspect therefore the invention provides a cationic lipid comprising a plurality of cationic moieties or cationic precursors within a plurality of headgroups, a lipophilic moiety and a linking moiety positioned between the lipophilic moiety and the headgroups, wherein the lipophilic moiety is connected to the linking moiety through a nitrogen-containing linkage and each of the plurality of cationic moieties or precursors is connected through an ester moiety to the linking moiety.

Viewed from a second aspect the invention provides a composition comprising a cationic lipid according to the first aspect of the invention in combination with an additional lipid.

Viewed from a third aspect the invention provides a composition comprising a cationic lipid according to the first or second aspects of the invention in combination with a polynucleotide.

Viewed from a fourth aspect the invention provides a method for transfecting a polynucleotide into a cell comprising contacting a cell with a composition according to the third aspect of this invention.

Viewed from a fifth aspect, the invention provides a kit of parts comprising a composition according to this invention and a cell into which the polynucleotide of the composition may be transfected.

### BRIEF DESCRIPTION OF THE DRAWINGS

FIG. 1 shows a synthetic scheme for biodegradable compounds 6a-n.

FIG. 2 shows a synthetic scheme for intermediate 11.

FIG. 3 shows a synthetic scheme for biodegradable compounds 13a,b and 15a,b.

FIG. 4 shows a synthetic scheme for biodegradable compounds 21a,b.

FIG. 5 shows flow cytometry analysis of HeLa cells 48 hours after transfection with a GFP (green fluorescent protein) reporter plasmid A) Untransfected cell control; B) Pentadecanoyl derivative 6f, N/P 12, 1:2 mol mixture with DOPE; C) Effectene® Transfection Reagent; D) Lipofectamine™ 2000.

FIG. 6 shows percentage of transfected cells (calculated by flow cytometry analysis) of HeLa cells 48 hours after transfection with a GFP-reporter plasmid. Compounds 6f, 6i, 6m, 13b, and 15a from the invention were assayed and compared with Lipofectamine™ 2000 and Effectene® Transfection Reagent.

FIG. 7 shows the results of a cell viability study performed 48 hours after transfection. Results are shown in respect of a control, five cationic lipids of this invention (6f, 6i, 6m, 13b, and 15a), Lipofectamine™ 2000 and Effectene® Transfection Reagent.

FIG. 8 shows flow cytometry analysis of an RNAi knock-down assay with GFP-expressing mES (mouse embryonic stem) cells after 48 h. A) Untransfected cell control; B) Lipofectamine™ 2000, C) Pentadecanoyl derivative 6f, N/P 12, 1:2 mol mixture with DOPE.

FIG. 9 shows the non-invasive in vivo luminescence imaging of anesthetized mice transfected with 16 μg of a luciferase-reporter plasmid (pLux) complexed with derivative 6i (N/P 12, 1:2 mol mixture with DOPE) (left mouse) and naked plasmid (right mouse) 72 hours after transfection. Mice were scanned 15 min after intraperitoneal administration of firefly luciferin (15 mg/kg) in the anesthetized mice.

FIG. 10 shows the same mice as in FIG. 9. Images were taken 120 hours after transfection. Images were captured 15 min after intraperitoneal administration of firefly luciferin (15 mg/kg) in the anesthetized mice and taken at 2 min intervals.

### DETAILED DESCRIPTION OF THE INVENTION

The present invention provides new cationic lipids with an architecture that is susceptible to degradation under physiological conditions to afford relatively non-toxic components. The degradation is facilitated in particular in the context of transfection into cells by the presence of the ester moieties that are susceptible to hydrolysis under mildly acidic conditions. This occurs during cytoplasm entry by lipoplexes during endocytosis as a consequence of the natural drop in pH that occurs in the endosome: whilst the endosomal pH is initially that of the extracellular medium (approximately 7.2 to 7.4) the pH is progressively lowered to approximately 5.0 by ATP-dependent proton pumps within the endosomal membrane. In addition ester bonds are susceptible to hydrolysis by intracellular lipases once endocytosis is complete.

Separately, the lipophilic component, which is joined to the linking moiety by a nitrogen-containing linkage, is susceptible to release by degradation at this linkage.

Advantageously, the linking moiety present in the cationic lipids of this invention, and from which the cationic lipids may be prepared, is a polyfunctional molecule with functionality appropriate to react with other molecules so as to provide the nitrogen-containing linkage and ester moieties in the cationic lipids. Thus the linking moiety may, for example, comprise a plurality of (i) hydroxyl or (ii) carboxylic acid groups, (in particular hydroxyl groups), which may react respectively with (i) a plurality of carboxylic acid and cationic headgroup-containing molecules; or (ii) a plurality of hydroxyl and cationic headgroup containing molecules, so as to introduce the plurality of cationic headgroups. It will also be understood that the carboxylic acids referred to in this context may be activated equivalents thereof, such as an acyl chloride, as is known in the art.

Separately the molecule from which the linking moiety is derived may comprise an amine (or other) moiety from which the nitrogen-containing linkage is derived.

Conveniently, we find that the very well known molecules 2-amino-2-hydroxymethyl-1,3-propanediol, commonly known and referred to herein as tris, and 2-amino-1,3-propanediol, commonly known and referred to herein as serinol, are two excellent building blocks from which to prepare the cationic lipids of this invention. In particular the tris or serinol core, which is revealed upon degradation of the nitrogen-containing linkage and ester moieties, is a soluble and substantially non-toxic moiety. Tris and serinol are efficiently removed by the kidneys in vivo. By the presence of the three hydroxyl groups in tris, cationic lipids comprising three distinct headgroups may be prepared in which the headgroups are connected to the tris skeleton by ester groups formed (in part) from these hydroxyl groups. Equivalently, cationic lipids comprising two distinct headgroups may be prepared in which the headgroups are connected to the serinol skeleton by ester groups formed (in part) from these hydroxyl groups. Whilst the remainder of the discussion below focuses in the main upon tris- and serinol-derived cationic lipids it is to be understood that this is not to be considered as limiting the invention but, simply, a convenient way to demonstrate certain embodiments of the invention as a consequence of the ready availability of tris and serinol. Other appropriate molecules from which the linking moiety present in the cationic lipids may be derived will be evident to those skilled in the art.

In tris-derived cationic lipids, as a consequence of the presence of three cationic headgroups—one attached to each of the hydroxyl oxygen atoms of the tris molecule by way of ester molecules derived from these oxygen atoms and keto groups contributed by linking moieties attached to the cationic headgroups—such lipids and others with three cationic headgroups have a tripod-like geometry in which the plane typically formed by the cationic headgroups is spatially disposed towards the remainder of the lipid such that it is perpendicular to the hydrophobic moiety or moieties that is or are the tail or tails of the lipid. This geometry in particular is believed to contribute to the packing properties of the amphiphilic lipid molecules where the molecules to be packed are DNA (or other polynucleotides). It is believed that this is achieved by way of enhanced interaction between the positively charged headgroups of the lipid and the negatively charged groups of the polynucleotide, which, in turn, promotes hydrophobic interactions between the hydrophobic tails. The resultant supramoleculecular lipoplex formation is believed to assist lipoplex formation.

The cationic moieties are typically derived from any basic nitrogen-based functional group capable of undergoing protonation at physiological pH. However, as is known in the art (see e.g. Martin B. et al. (infra)) other cationic groups such as phosphonium and arsonium moieties may also be used. Whilst the ensuing discussion focuses on nitrogen-based moieties as the cationic species, the present invention is not to be considered to be so limited. The susceptibility of nitrogen-based and other groups to protonation is the reason for the use herein of the term cationic precursors: cationic precursors are functional groups that can provide cationic moieties by undergoing protonation at physiological pH, or by quaternisation, for example. Given that it is the cationic moieties themselves that are useful in most applications, the following discussion focuses primarily upon these.

Typically the headgroups are protonated amine or guanidine groups, these groups being protonated at physiological pH. Guanidine groups (—NH—C(═O—NH)NH₂) are strongly basic and are thus attractive to use as the moieties from which cationic headgroups are derived because of the pH insensitivity towards the formation of the desired, protonated guanidinium moieties. The guanidine group is found for example in the natural cationic amino acid arginine.

Alternatively the cationic moieties may be derived from amines, such as primary, secondary, tertiary or even quaternary (i.e. permanently charged) amines. Typically the cationic moieties will constitute a protonated primary amine although, as is known in the art, quaternary amines, or protonated secondary and tertiary amines, may also be used as the cationic headgroups. With secondary, non-primary amines the amine may be contained within a linking moiety that is connected to the tris-based cationic lipid via an ester linkage. With tertiary or quaternary amines the amine will typically have one to three straight-chain or branched alkyl groups attached to the nitrogen atom. Typically there will be selected from C₁₋₁₀ alkyl groups, e.g. methyl, ethyl, or iso- or n-propyl, optionally substituted with one or more substituents selected from hydroxyl, mercapto, amino, keto, ester, amido etc.

The cationic headgroups, which may be the same or different, are connected to the linking moiety via ester groups, e.g. to the three oxygen atoms of the tris, serinol or other molecule via ester groups. Connecting the ester group and the cationic headgroup is a further linking moiety. This is typically a straight-chain or branched hydrocarbon chain (e.g. a C₁₋₃₀, more typically C₁₋₅, alkyl chain), that is to say the keto moiety within the ester group is considered not to be part of the linking moiety. The hydrocarbon chain may comprise one or more straight-chain, branched, or cyclic regions which may contain no or one or more heteroatoms selected from the group comprising oxygen, sulfur and nitrogen, and which may be unsubstituted or substituted either internally or externally with one or more heteroatoms or functional groups, e.g. one or two functional groups selected from the group comprising hydroxyl, oxo, mercapto, thio, sulfoxy, sulfonyl, amino, carboxy, keto and ester. In other embodiments the cyclic regions may comprise 1,4-phenylene or 1,4-dicyclohexylene.

Conveniently we find that the cationic headgroups may be attached to tris or other linking moiety by way of reaction between tris or other linking moiety, protected if appropriate, and molecules which contain the desired cationic headgroup, or molecules such as primary amino which may be easily converted to the desired cationic headgroup, e.g. by protonation. Such molecules are readily available to those skilled in the art. For example, molecules which may be reacted with tris, serinol or other molecules from which the linking moiety may be derived include amino acids or amino acyl derivatives thereof in which the amino group (e.g. terminal amino) is usually protected. Appropriate protection may be achieved by way of ^{t}Boc protection; other appropriate protecting groups will be known to those skilled in the art. Thus, as explained, a variety of amino- and carboxylic acid-containing molecules may be used in accordance with the present invention. These include γ-aminobutyric acid, γ-guanidinobutyric acid, 6-aminohexanoic acid, 6-guanidinohexanoic acid and natural amino acids (in addition to arginine already discussed above) such as glycine and other α-amino acids as well as β-alanine, the only naturally occurring β-amino acid.

More generally, any aminoacyl fragment may be attached to the oxygen atoms of the tris or other molecule to form the ester groups, for example α-aminoacyl, β-aminoacyl, γ-aminoacyl, δ-aminoacyl, and ε-aminoacyl fragments. Specific examples of such aminoacyl moieties include glycinyl, β-alanyl, γ-aminobutyryl, 5-aminopentanoyl, 6-aminohexanoyl, γ-guanidinobutyryl, 6-guanidinohexanoyl, lysinyl and arginyl. Glycinyl, β-alanyl and γ-aminobutyryl, are derived from glycine, β-alanine and GABA (γ-aminobutyric acid) respectively.

The cationic lipids of this invention are typically provided as salts of physiologically tolerable or pharmaceutically acceptable anions, optionally in aqueous media. Counteranions to the protonated amino or guanidine moieties or, where used, quaternary amino cations, are not particularly limited. Appropriate anions include halide anions such as fluoro, iodo, bromo and chloro, acetate, trifluoroacetate, bisulfate or methyl sulfate.

Alternatively, the cationic lipids may be generated simply by contacting their unprotonated precursors, if appropriate, with aqueous media. The resultant protonation serves to provide the desired cationic lipids.

Essentially, by definition, and because of the presence of the hydrophobic, also referred to as lipophilic portion, lipids, including the cationic lipids of the present invention, are not considered to be soluble in water. Thus emulsions, rather than solutions, result when the cationic lipids of this invention are contacted with aqueous media. The cationic lipids of this invention are indeed typically supplied or prepared as aqueous emulsions, which allow preparation of lipoplexes or other compositions by aliquoting a desired volume of the emulsion.

The aqueous media with which the cationic lipids of this invention form emulsions may be water. More typically, however it is a solution such as a saline solution (e.g. comprising 100-200 mM NaCl; or a buffered solution such as phosphate-buffered saline (PBS)). PBS typically comprises a mixture of dibasic and monobasic phosphates at a pH of about 7.0 to 7.6 (typically about 7.4) with NaCl at a concentration appropriate to make the resultant solution isotonic with the media, e.g. cell suspension, with which it is to be contacted. Other aqueous media with which it may sometimes be desired to mix the cationic lipids of this invention include any culture medium, typically a serum-free culture medium. Such culture media are known to those skilled in the art and are both easy to prepare and commercially available. They include DMEM (Dulbecco/Vogt modified Eagle's minimal essential medium) and RPMI (Rosswell Park Memorial Institute medium).

With regard to said lipophilic moiety one or two such moieties may be attached to the linking moiety, e.g. by a nitrogen-containing linkage as is now described.

Thus the lipophilic moiety may generally be a lipophilic motif of formula (I):

(D-E)ₙ-A- (I)

wherein:
n is 1 where A is absent or divalent and 2 where A is trivalent;
D is a saturated or unsaturated hydrocarbon moiety comprising from 1 to 100 carbon atoms, more usually 10 to 30 carbon atoms, for example 12 to 24 carbon atoms, which comprises one or more straight-chain, branched, or cyclic regions, which moiety may contain no or one or more heteroatoms selected from the group comprising oxygen, sulfur and nitrogen, and which moiety may be unsubstituted or substituted either internally or externally with one or more, e.g. one or two, functional groups selected from the group comprising hydroxyl, oxo, mercapto, thio, sulfoxy, sulfonyl, amino, carboxy, keto and ester;
E is absent or is selected from the group comprising carbonyl, amine, ether, thioether, phosphatidyl, sulfonyl, sulfoxide, ester, carbamoyl, carbamate and amide; and
A is absent or is a di or trivalent of one of the following formulae:

-G-D-G-, (-D-G)₂-J-G-D-G-, -G-D(G-)-K-G-D-G-

or (-G-D)-D(G-)-K-E-D-G-D-G-,

in particular -G-D-G-, (-D-G)₂-J-G-D-G-, - or (-G-D)-D(G-)-K-E-D-G-D-G-, wherein
each D and E is independently as hereinbefore defined;
each G is independently selected from the group comprising carbonyl, amine, ether, thioether, phosphatidyl, sulfonyl, sulfoxide, ester, carbamoyl, carbamate, amide and urea;
J is N or CH; and
K is NH or CH₂.

For example the moiety A where present in the above-described lipophilic moieties may be of the formulae -G-D-G-, (-D-G)₂-J-G-D-G- or -G-D(G-)-K-G-D-G-; or -G-D-G- or (-D-G)₂-J-G-D-G-, as hereinbefore defined.

In certain embodiments of formula (I) A is absent.

In certain embodiments of formula (I) E is absent. In such and other embodiments D-E- comprises a saturated or unsaturated fatty alkyl chain. For example, D- may be decyl, undecyl, dodecyl, tridecyl, tetradecyl, pentadecyl, hexadecyl, octadecyl, 9-octadecenyl (also known as oleyl), eicosyl or tetraeicosyl; or may be generally represented as CH₃(CH₂)ₚ— wherein p is from 5 to 100, more usually 10 to 30, for example 12 to 24.

In certain embodiments of formula (I) A is absent and E is present and is a carbonyl, amide or ester. In these embodiments E and a nitrogen atom may form the nitrogen-containing linkage as an amide, urea or carbamate moiety. Typically the nitrogen-containing linkage of the cationic lipids of these and other embodiments of the invention is an amide moiety.

In certain embodiments of formula (I) D-E- is a saturated or unsaturated fatty acyl chain. For example, D-E- may be decanoyl, undecanoyl, dodecanoyl, tridecanoyl, tetradecanoyl, pentadecanoyl, hexadecanoyl, octadecanoyl, 9-octadecenoyl, eicosanoyl or tetraeicosanoyl; or may be represented as CH₃(CH₂)_{q}C(═O)— wherein q is from 5 to 100, more usually 10 to 30, for example 12 to 24.

In certain embodiments of formula (I), D may comprise fused cyclic regions whereby to form a polycyclic hydrocarbyl moiety. An example of a D-E- moiety comprising such a D- is cholesteryloxycarbonyl. In other embodiments the cyclic regions may comprise 1,4-phenylene or 1,4-dicyclohexylene.

In certain embodiments of formula (I) where A is present, and is a moiety of formula -G-D-G-, each G may be carbonyl or amide. Alternatively each G may be amine. In these and other embodiments D may be an alkylene moiety of the formula —(CH₂)ₙ— wherein r is from 1 to 10, e.g. 1 to 6. Advantageously such alkylene moieties may be obtained from readily available, and substantially non-toxic, starting materials such as GABA (γ-aminobutyric acid) or AHX (6-aminohexanoic acid) and succinic acid. In these and other embodiments D-E- may be a saturated or unsaturated fatty acyl chain (when GABA or AHX is present) or fatty alcohol (when succinic acid is present). For example, D-E- may be decanoyl or decyloxy, undecanoyl or undecyloxy, dodecanoyl or dodecyloxy, tridecanoyl or tridecyloxy, tetradecanoyl or tetradecyloxy, pentadecanoyl or pentadecyloxy, hexadecanoyl or hexadecyloxy, octadecanoyl or octadecyloxy, 9-octadecenoyl or 9-octadecenyloxy, eicosanoyl or eicosyloxy, tetraeicosanoyl or tetraecosyloxy; or may be represented as CH₃(CH₂)_{q}C(═O)— or CH₃(CH₂)_{q}C—O— wherein q is from 5 to 100, more usually 10 to 30, for example 12 to 24.

In certain embodiments of formula (I) where A is present, and is a moiety of formula (-D-G)₂-J-G-D-G-, the (-D-G)₂-J- moiety may be (—O—CH₂)₂—CH—O— and so derived from glycerol. In these and other embodiments each G may be carbonyl or amide and D may be an alkylene moiety of the formula —(CH₂)ᵣ— as hereinbefore defined. Advantageously such moieties may be obtained from readily available, and substantially non-toxic, starting materials such as GABA (γ-aminobutyric acid) and succinic acid.

In certain embodiments of formula (I) where A is present, and is a moiety of formula -G-D(G-)-K-G-D-G-, the -G-D(G-)-K- moiety may be —O—C(═O)—(CH₂)ᵣ—CH(C(═O)(—O—))—NH— which is derived from aminoacids such as glutamic or aspartic acid. In these and other embodiments each G in the -G-D-G- moiety may be carbonyl or amide and D may be an alkylene moiety of the formula —(CH₂)ᵣ— as hereinbefore defined. Thus moieties of formula -G-D(G-)-K-G-D-G- may be degraded into relatively non-toxic materials such as glutamic acid or aspartic acid, and succinic acid.

In certain embodiments of formula (I) where A is present, and is a moiety of formula (-G-D)-D(G-)-K-E-D-G-D-G-, the (-G-D)-D(G-)-K-E-D- moiety may be (—OCH₂)—CH(O—)—CH₂—O—P(═O)(—O⁻)—O—(CH₂)₂—NH— which is derived from phosphatidylethanolamines such as DOPE. In these and other embodiments each G in the -G-D-G- moiety may be carbonyl or amide and D may be an alkylene moiety of the formula (CH₂)ᵣ as hereinbefore defined. Thus moieties of formula (-G-D)-D(G-)-K-E-D-G-D-G- may be degraded into relatively non-toxic materials such as DOPE, and γ-aminobutyric acid or succinic acid.

The compounds of this invention are made, in broad terms, by reacting a molecule that provides the linking moiety of the cationic lipid, e.g. tris or serinol, with other molecules so as to introduce the required lipophilic tail(s) and cationic headgroups. Typically the tris or other molecule is initially reacted to introduce the lipophilic tail(s) or any linking motifs as described hereinbefore. In order to do this the hydroxyl groups are advantageously protected so as to allow selective functionalisation of the primary amine. We find the tert-butyldimethylsilyl (TBDMS) protection group appropriate although other groups will be evident to those skilled in the art.

After protection of the hydroxyl groups, the nitrogen-containing linkage may be introduced. As an example of how this is achieved, where the linking moiety is derived from tris, its primary amine will be functionalised. In certain embodiments of the invention the linking moiety is attached to a single lipophilic tail; as described above this may be derived from a saturated or unsaturated fatty acid, typically having from 10-24 carbon atoms, more typically 12 to 18 carbon atoms, and achieved by the reaction with a corresponding activated fatty acid, such as an acyl chloride, or by a DCC/DMAP activated derivative. Examples of fatty acyl chains which may be attached to the amino group of tris, whereby to form amide linkages are decanoyl, undecanoyl, dodecanoyl, tridecanoyl, tetradecanoyl, pentadecanoyl, hexadecanoyl, octadecanoyl, 9-octadecenoyl, eicosanoyl, tetraeicosanoyl, or cholesteryloxicarbonyl. In one particular embodiment of the invention the fatty acyl chain is oleyl (9-octadecenoyl), which is derived from oleic acid. Alternatively the lipophilic tail may be defined in accordance with one of the other possibilities described above which may likewise be introduced onto the tris or other molecule that provides the linking moiety using chemical methodologies known to those skilled in the art.

As will be appreciated from the foregoing, the cationic lipids of the present invention are of particular use in the transfection of polynucleotides, such as DNA and RNA, and in particular DNA and siRNA, into cells. However, the utility of the cationic lipids of the present invention is not limited to their use in the preparation of lipoplexes for delivery of RNA and DNA into cells. Rather, the cationic lipids of the present invention may be used to envelope other macromolecules, such as proteins or polypeptides, for introduction into cells, or indeed to envelope small chemical compound such as synthetic pharmaceuticals, or complexing metal ions, e.g. tripositive metal ions such as Fe(III), Ga(III), Ge(III), or Cr(III); or Fe(III), Gd(III), Eu(III), Ge(III) or Cr(III), which are used in diagnostic contrast agents. Nevertheless, the particular affinity of cationic lipids for anionic compounds, including polyanionic compounds such as nucleic acids, is the use to which cationic lipids are most suitable, and the utility of the cationic lipids of the invention in the preparation of lipoplexes comprising DNA and RNA is the use focused on herein.

The cationic lipids of the present invention may be used for transfection or other purposes, either in the presence or in the absence of co-helper lipids. They may be supplied either dried, or as aqueous emulsions as hereinbefore described. Such co-helper lipids, as is described hereinbefore, are typically neutral lipids and include DOPE, 1,2-dioleoyl-glycero-3-phosphocholine (DOPC) and cholesterol.

Where present, a co-helper lipid may be present in a wide range of ratios vis-á-vis the cationic lipid of this invention, for example molar ratios of about 1:10 to 10:1 of co-helper lipid:cationic lipid may be used, more usually from about 1:5 to 5:1, e.g. about 3:1 to 1:1. For example we find a 2:1 mixture comprising 2 molar equivalents of DOPE to a cationic lipid of this invention to be a generally useful ratio to use.

Where used for transfection, of RNA (e.g. siRNA) or DNA, the cationic lipid is typically present in excess over the amount of RNA or DNA present. For example the cationic lipid may be present in excesses of about 2 to 100:1, vis-á-vis the amount of DNA or RNA, e.g. about 2 to 50:1, typically between about 5:1 and 25:1. These ratios relate to the ratio of positive charges to negative charges. The positive charges are provided by the cationic moieties present in the cationic lipids of this invention and the negative charges are provided by the ionised phosphate groups or other negative charges present in the polynucleotide, for example DNA or RNA. Thus, for example, if it is desired to form a lipoplex with a DNA comprising 510 phosphate groups, a five-times excess of tris-derived cationic lipid of this invention, wherein each of the three hydroxyl moieties is derivatised with a cationic headgroup comprising one cation, requires 170 molar equivalents of such a cationic lipid to provide a number of cations (3×170) equivalent to match those in the DNA and 850 molar equivalent would be necessary to provide a five-times excess. The ratios of the charges provided by the cationic lipids to the charges provided by polynucleotides are referred to herein as N/P ratios. We find useful N/P ratios with which to work, e.g. form lipoplexes, to be those in the range of 2/1 to 60/1, for example 3/1 to 50/1, e.g. 5/1 to 30/1. Useful ratios reported herein are 6/1, 12/1 and 24/1. The N/P ratios referred to herein may also be used when using the quantities of co-helper lipids described herein, particularly in the immediately preceding paragraph.

The method of the present invention may be applied to in vitro and in vivo transfection of cells, particularly to transfection of eukaryotic cells or tissue such as animal cells, in particular mammalian cells, in particular human cells as well as other cells such as those of insects, plant, birds and fish.

The method of the invention can thus be used to generate transfected cells or tissues capable of expressing useful gene products as a result of the DNA or RNA, in particular DNA, transfected, as well as having utility in the field of biotechnology and medical research, gene therapy and other therapeutic applications, either in vivo or ex vivo. Such therapeutic applications include cancer treatment, and in diagnostic methods.

The compounds of the invention are of particular use in the transfection of RNA and DNA into cells. Whilst the transfection into cells of any polynucleotide may be advantageous, transfection of plasmid DNA, optionally modified to provide antibiotic resistance, and the delivery of siRNA (small interfering RNA) are of particular utility in relation to biotechnological applications (e.g. in vitro gene transfection or gene silencing) and in gene therapy.

To demonstrate the utility of the cationic lipids of the present invention as in vitro transfection vectors, as is described in greater detail in the experimental section below, we demonstrate transfection into human tumoral HeLa cells of a plasmid DNA encoding for GFP, and a siRNA responsible for the knocking-down of stable GFP-biosynthesis from GFP-expressing mES cells. Our results demonstrate both effective transfection of the polynucleotides and satisfactory toxicity data towards the HeLa cells transfected.

To demonstrate the utility of the cationic lipids of the present invention as in vivo transfection vectors, as is described in greater detail in the experimental section below, we demonstrate transfection of a plasmid DNA encoding for luciferase by instillation into mouse lung. Our results demonstrate effective transfection of the lung and other organs, with no discernable toxic effects on animal health.

The compositions described herein can be used to transfect a variety of polynucleotides, such as plasmid DNA, viral DNA, chromosomal fragments, antisense oligonucleotides, antisense phosphorothioate oligonucleotides, RNA molecules and ribozymes, or combinations thereof.

The transfection methods can be performed in vitro, e.g., wherein the transfection composition is applied to cells in culture. Alternatively, the methods can be performed in vivo by applying the transfection composition to cells in vivo.

As used herein, the various forms of the term “transfect” (e.g., “transfecting”, “transfected”) are intended to refer to the process of introducing a polynucleotide molecule from an exterior location into the interior of a cell.

As used herein, the term “polynucleotide molecule” is intended to encompass molecules comprised of two or more covalently linked nucleotide bases, including deoxyribonucleic acid (DNA) molecules and ribonucleic acid (RNA) molecules. The nucleotides forming the polynucleotide molecule typically are linked to each other by phosphodiester linkages, although the term “polynucleotide molecule” is also intended to encompass nucleotides linked by other linkages, such as phosphorothioate linkages. Nonlimiting examples of polynucleotide molecules include plasmid DNA, viral DNA, chromosomal fragments, antisense oligonucleotides, antisense phosphorothioate oligonucleotides, RNA molecules (read as siRNA molecules) and ribozymes.

For gene therapy purposes, the polynucleotide(s) typically is an expression vector (described in further detail below) that encodes a protein to be provided for therapeutic benefit. The transfection method preferably is used to transfect eukaryotic cells, more preferably mammalian cells. The transfection method can be carried out in vitro, e.g., by applying the transfection composition to cells in culture. The time period for contacting the transfection composition with the cells in culture can be optimized by standard methods. A nonlimiting example of a transfection time in vitro is 48 hours, followed by washing the cells (e.g., with phosphate buffered saline). Alternatively, the transfection method can be carried out in vivo, by applying the transfection composition to cells in vivo. Typical target tissues for transfection in vivo include, for example, stomach, muscle, lungs, liver, epithelial cells, colon, uterus, intestine, heart, kidney, prostate, skin, eye, brain, penile tissue and nasal tissue.

In certain embodiments the polynucleotide may be in the form of an expression vector encoding a protein(s) of therapeutic benefit. An expression vector comprises a polynucleotide in a form suitable for expression of the polynucleotide in cells to be transfected, which means that the recombinant expression vector includes one or more regulatory sequences, usually selected on the basis of the type of cells to be transfected, which is operatively linked to the polynucleotide to be expressed. Within a recombinant expression vector, “operably linked” is intended to mean that the polynucleotide of interest is linked to the regulatory sequence(s) in a manner which allows for expression of the polynucleotide (e.g., transcription/translation in a host cell when the vector is introduced into the host cell). The term “regulatory sequence” is intended to includes promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a polynucleotide in many types of host cell and those which direct expression of the polynucleotide only in certain host cells (e.g. tissue-specific regulatory sequences). It will be appreciated by those skilled in the art that the design of the expression vector may depend on such factors as the choice of the host cell to be transformed, the level of expression of protein desired, etc.

Examples of mammalian expression vectors include pMex-NeoI, pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. Alternatively, mammalian expression vectors capable of directing expression of a polynucleotide preferentially in a particular cell type can be used (i.e., an expression vector comprising tissue-specific regulatory elements) and are well known in the art.

The transfection methods of the present invention employing the compounds or compositions (such as those described above) of the present invention or mixtures thereof can be applied to in vitro and in vivo transfection of cells, particularly to transfection of eukaryotic cells or tissues including animal cells, human cells, insect cells, plant cells, avian cells, fish cells, mammalian cells and the like.

The methods of this invention can be used to generate transfected cells or tissues which express useful gene products. The methods of this invention can also be used as a step in the production of transgenic animals. The methods of this invention are useful in any therapeutic method requiring introducing of nucleic acids into cells or tissues. In particular, these methods are useful in cancer treatment, in in vivo and ex vivo gene therapy, and in diagnostic methods. See, for example, U.S. Pat. No. 5,589,466 to Feigner, et al. and U.S. patent application Ser. No. 08/450,555 filed on May 25, 1995 to Jessee, et al. The transfection compounds or compositions of this invention can be employed as research reagents in any transfection of cells or tissues done for research purposes. Nucleic acids that can be transfected by the methods of this invention include DNA and RNA from any source comprising natural bases or non-natural bases, and include those encoding and capable of expressing therapeutic or otherwise useful proteins in cells or tissues, those which inhibit expression of nucleic acids in cells or tissues, those which inhibit enzymatic activity or activate enzymes, those which catalyze reactions (ribozymes), and those which function in diagnostic assays.

The compounds, compositions and methods provided herein can also be readily adapted in view of the disclosure herein to introduce biologically active macromolecules or substances other than nucleic acids, including, among others, polyamines, polyamine acids, polypeptides, proteins, biotin, and polysaccharides into cells. Other useful materials for example, therapeutic agents, diagnostic materials and research reagents, can be introduced into cells by the methods of this invention. In a preferred aspect, any nucleic acid vector may be delivered to or into a cell by the present invention.

This invention also includes transfection kits which include one or more of the compounds or compositions of the present invention or mixtures thereof. Particularly, the invention provides a kit comprising one or more of the compounds of the present invention and at least one additional component selected from the group consisting of a cell, cells, a cell culture media, a nucleic acid, a transfection enhancer and instructions for transfecting a cell or cells.

The polynucleotide molecules used in the present invention may be either single-stranded or double-stranded, may be linear or circular, e.g., a plasmid, and are either oligo- or polynucleotides. They may comprise as few as 15 bases or base pairs, or may include as many as 20 thousand bases or base pairs (20 kb). Since the transfer moiety is employed on a pro rata basis when added to the nucleic acid composition, practical considerations of physical transport will largely govern the upper limit on the size of nucleic acid compositions which can be utilized.

In addition to these naturally occurring materials, the nucleic acid compositions used in the present invention can also include synthetic compositions, i.e., nucleic acid analogs. These have been found to be particularly useful in antisense methodology, which is the complementary hybridization of relatively short oligonucleotides to single-stranded RNA or single-stranded DNA, such that the normal, essential functions of these intracellular nucleic acids are disrupted. See, e.g., Cohen, Oligonucleotides: Antisense Inhibitors of Gene Expression, CRC Press, Inc., Boca Raton, Fla. (1989).

The size, nature and specific sequence of the nucleic acid composition to be transferred to the target cell can be optimized for the particular application for which it is intended, and such optimization is well within the skill of the artisan in this field.

The polynucleotide molecules may serve as: 1) genetic templates for proteins that function as prophylactic and/or therapeutic immunizing agents; 2) replacement copies of defective, missing or non-functioning genes; 3) genetic templates for therapeutic proteins; 4) genetic templates for antisense molecules and as antisense molecules per se; or 5) genetic templates for ribozymes.

In the case of polynucleotide molecules which encode proteins, the polynucleotide molecules preferably comprise the necessary regulatory sequences for transcription and translation in the target cells of the individual animal to which they are delivered.

In the case of polynucleotide molecules which serve as templates for antisense molecules and ribozymes, such nucleic acid molecules may be linked to regulatory elements necessary for production of sufficient copies of the antisense and ribozyme molecules encoded thereby respectively.

The present invention can allow for transfer to target cells of a polynucleotide molecule that comprises a nucleotide sequence that either encodes a desired peptide or protein, or serves as a template for functional nucleic acid molecules. The desired protein or functional nucleic acid molecule may be any product of industrial, commercial or scientific interest, e.g., therapeutic agents including vaccines; foodstuffs and nutritional supplements; compounds of agricultural significance such as herbicides and plant growth regulants, insecticides, miticides, rodenticides, and fungicides; compounds useful in animal health such as parasiticides including nematocides; and so forth. The target cells are typically cultures of host cells comprising microoganism cells such as bacteria and yeast, but may also include plant and mammalian cells. The cell cultures are maintained in accordance with fermentation techniques well known in the art, which maximize production of the desired protein or functional nucleic acid molecule, and the fermentation products are harvested and purified by known methods.

The present invention further relates to a method for the transfer of a polynucleotide molecule composition to the cells of an individual in an in vivo manner.

The nucleic acid molecule may be administered to the cells of said individual on either an in vivo or ex vivo basis, i.e., the contact with the cells of the individual may take place within the body of the individual in accordance with the procedures which are most typically employed, or the contact with the cells of the individual may take place outside the body of the individual by withdrawing cells which it is desired to treat from the body of the individual by various suitable means, followed by contacting of said cells with said nucleic acid molecule, followed in turn by return of said cells to the body of said individual.

The method of transferring a polynucleotide composition to the cells of an individual provided by the present invention, includes particularly a method of immunizing an individual against a pathogen. In this method, the polynucleotide composition administered to said cells, comprises a nucleotide sequence that encodes a peptide which comprises at least an epitope identical to, or substantially similar to an epitope displayed on said pathogen as antigen, and said nucleotide sequence is operatively linked to regulatory sequences. The nucleic acid molecule must, of course, be capable of being expressed in the cells of the individual.

The method of transferring a polynucleotide composition to the cells of an individual provided by the present invention, further includes methods of immunizing an individual against a hyperproliferative disease or an autoimmune disease. In such methods, the polynucleotide composition which is administered to the cells of the individual comprises a nucleotide sequence that encodes a peptide that comprises at least an epitope identical to or substantially similar to an epitope displayed on a hyperproliferative disease-associated protein or an autoimmune disease-associated protein, respectively, and is operatively linked to regulatory sequences. Here again, the nucleic acid molecule must be capable of being expressed in the cells of the individual.

Other aspects of the present invention relate to gene therapy. This involves compositions and methods for introducing polynucleotide molecules into the cells of an individual which are exogenous copies of genes which either correspond to defective, missing, non-functioning or partially functioning genes in the individual, or which encode therapeutic proteins, i.e., proteins whose presence in the individual will eliminate a deficiency in the individual and/or whose presence will provide a therapeutic effect on the individual. There is thus provided a means of delivering such a protein which is a suitable, and even preferred alternative to direct administration of the protein to the individual.

As used herein the term “desired protein” is intended to refer to peptides and proteins encoded by gene constructs used in the present invention, which either act as target proteins for an immune response, or as a therapeutic or compensating protein in gene therapy regimens.

Using the methods and compositions of the present invention, DNA or RNA that encodes a desired protein is introduced into the cells of an individual where it is expressed, thus producing the desired protein. The nucleic acid composition, e.g., DNA or RNA encoding the desired protein is generally linked to regulatory elements necessary for expression in the cells of the individual. Regulatory elements for DNA expression include a promoter and a polyadenylation signal. In addition, other elements, such as a Kozak region, may also be included in the polynucleotide composition.

Examples of promoters useful with the nucleic acid compositions used in the present invention, especially in the production of a genetic vaccine for humans, include but are not limited to, promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV), as well as promoters from human genes such as human Actin, human Myosin, human Hemoglobin, human muscle creatine and human metalothionein.

Examples of polyadenylation signals useful with the nucleic acid compositions used in the present invention, especially in the production of a genetic vaccine for humans, include but are not limited to, SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, may be used.

In addition to the regulatory elements required for nucleic acid molecule expression, other elements may also be included in the DNA molecule. Such additional elements include enhancers. The enhancer may be selected from the group including but not limited to: human Actin, human Myosin, human Hemoglobin, human muscle creatine, and viral enhancers such as those from CMV, RSV and EBV.

Nucleic acid compositions can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region, which produces high copy episomal replication without integration. In aspects of the invention relating to gene therapy, constructs with origins of replication including the necessary antigen for activation are preferred.

Antisense molecules and ribozymes may also be delivered to the cells of an individual by introducing a nucleic acid composition which acts as a template for copies of such active agents. These agents inactivate or otherwise interfere with the expression of genes that encode proteins whose presence is undesirable. Nucleic acid compositions which contain sequences that encode antisense molecules can be used to inhibit or prevent production of proteins within cells. Thus, production of proteins such as oncogene products can be eliminated or reduced. Similarly, ribozymes can disrupt gene expression by selectively destroying messenger RNA before it is translated into protein. In some embodiments, cells are treated according to the invention using nucleic acid compositions that encode antisense or ribozymes as part of a therapeutic regimen which involves administration of other therapeutics and procedures. Polynucleotide compositions encoding antisense molecules and ribozymes use similar vectors as those which are used when protein production is desired except that the coding sequence does not contain a start codon to initiate translation of RNA into protein.

Ribozymes are catalytic RNAs which are capable of self-cleavage or cleavage of another RNA molecule. Several different types of ribozymes, such as hammerhead, hairpin, Tetrahymena group I intron, ahead, and RNase P are known in the art; see S. Edgington, Biotechnology (1992) 10, 256-262. Hammerhead ribozymes have a catalytic site which has been mapped to a core of less than 40 nucleotides. Several ribozymes in plant viroids and satellite RNAs share a common secondary structure and certain conserved nucleotides. Although these ribozymes naturally serve as their own substrate, the enzyme domain can be targeted to another RNA substrate through base-pairing with sequences flanking the conserved cleavage site. This ability to custom design ribozymes has allowed them to be used for sequence-specific RNA cleavage; see G. Paolella et al., EMBO (1992), 1913-1919.) It will therefore be within the skill of one in the art to use different catalytic sequences from various types of ribozymes, such as the hammerhead catalytic sequence, and design them in the manner disclosed herein. Ribozymes can be designed against a variety of targets including pathogen nucleotide sequences and oncogenic sequences. Preferred embodiments include sufficient complementarity to specifically target the abl-bcr fusion transcript while maintaining efficiency of the cleavage reaction.

The present invention also provides pharmaceutical kits which comprise a container comprising a polynucleotide composition, and a container comprising a transfection agent of the present invention. Optionally, there is included in such kits excipients, carriers, preservatives and vehicles suitable pharmaceutical compositions and known to those skilled in the art. The term pharmaceutical kit is also intended to include multiple inoculants used in the methods of the present invention. Such kits include separate containers comprising different inoculants and transfection agents. The pharmaceutical kits in accordance with the present invention are also contemplated to include a set of inoculants used in immunizing methods and/or therapeutic methods, as described above.

The invention is now illustrated by the non-limiting examples that follow.

### 1. General Remarks

TLC was performed on silica plates using varying systems as stated. Plates were visualised under an UV lamp at 254 nm or by a ninhydrin test.

¹H, ¹³C and DEPT NMR spectra were recorded on a Bruker AC300 spectrometer operating at 250 MHz for ¹H and 62.5 MHz for ¹³C and DEPT at 298K. Samples were dissolved in CDCl₃ or DMSO-d⁶ or methanol-d as stated. Chemical shifts are quoted in parts per million relative to the solvent peak. Abbreviations for multiplicity are s, singlet, d, doublet, t, triplet, q, quadruplet and m, multiplet. All coupling constants were measured in Hz.

Electrospray-mass spectroscopy spectra were recorded using an Agilent 1100 series VG platform Quadruple Electrospray Ionisation mass spectrometer model G1946B. Sonification was done using a Hilsonic water bath and flow cytommetry using a BD FACS Aria flow cytometer.

In vivo imaging of luminescence was carried out using an IVIS SPECTRUM Imaging System (Caliper).

Organic solvents were supplied by Fisher Scientific and laboratory reagents by Sigma-Aldrich.

### 2. Synthesis of the Biodegradable Compounds

2.1. Compounds with General Structure:

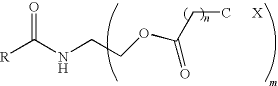

In particular compounds with this general structure and having:
R—C(═O)— a fatty acyl chain such as decanoyl, undecanoyl, dodecanoyl, tridecanoyl, tetradecanoyl, pentadecanoyl, hexadecanoyl, octadecanoyl, 9-octadecenoyl (oleyl), eicosanoyl, or tetraeicosanoyl.
n=1, 2, 3, 4 or 5
m=2 or 3

### C=NH₂ or —NH—C(═NH)NH₂

### X=CF₃—C(═O)OH or HCl

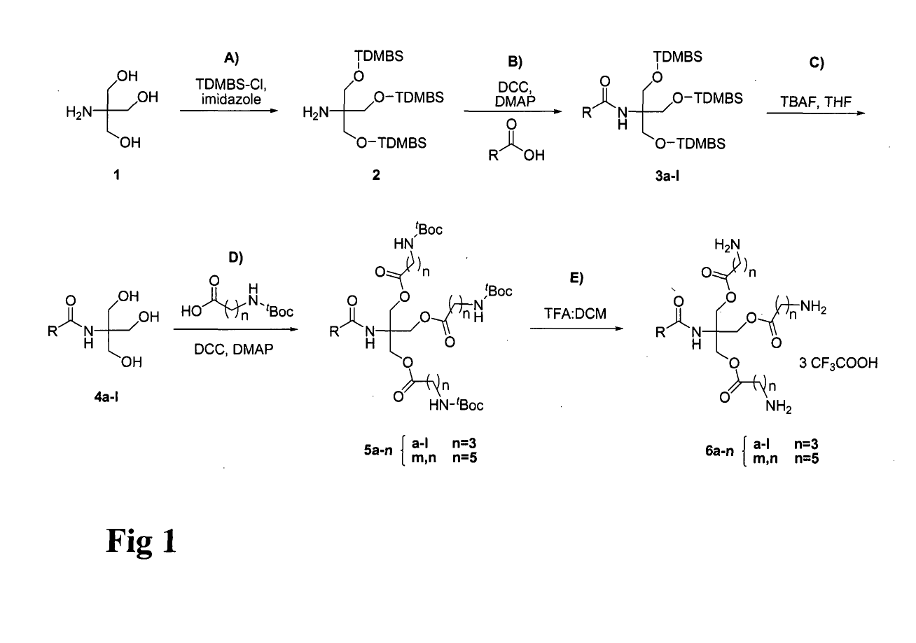

Synthesis of a library of cationic lipids 6a-n the invention was achieved by following the synthetic strategy, outlined in the scheme shown in FIG. 1 and described below together with details of selected examples. The groups R—C(═O)— are as follows for each compound: 6a decanoyl, 6b undecanoyl, 6c dodecanoyl, 6d tridecanoyl, 6e tetradecanoyl, 6f pentadecanoyl, 6g hexadecanoyl, 6h octadecanoyl, 6i 9-octadecenoyl, 6j eicosanoyl, 6k tetraeicosanoyl, 6l cholesteryloxycarbonyl, 6m pentadecanoyl, 6n 9-octadecenoyl.

### A) Synthesis of tris-(tert-butyldimethylsilyloxymethyl)aminomethane, 2

Butyldimethylsilyl chloride (6.7 g, 44.6 mmol) and imidazole (6.3 g, 92.9 mmol) were dissolved in DMF (5 mL). Tris(hydroxymethyl)methyl amine, 1 (1.5 g, 12.4 mmol) was added and the mixture stirred at room temperature for 1 hour. The product was washed with H₂O, extracted with DCM, dried over anhydrous MgSO₄ and filtered. Solvent was removed under reduced pressure to give a white powder (5.6 g, 12.1 mmol, 98%): ¹H NMR (CDCl₃) δ: 3.4 (s, 6H), 0.85 (s, 27H), 0.0 (s, 18H). ¹³C NMR (CDCl₃) δ: 64.5, 57.1, 26.3, 17.9. LRMS (ES+) m/z 464.5 [100, (M+H⁺)].

### B) Synthesis of Fatty Derivatives 3a-l

Method A. Either decanoyl chloride or cholesteryl chloroformate was added dropwise into a solution of compound 2 (1 equiv.), pyridine (1.1 equiv.) and dimethylaminopyridine (DMAP, 0.1 equiv.) in DCM and stirred for 2 h. Subsequently, the solution was filtered and the solvent removed under reduced pressure and the crude product redissolved in DCM (three times). The product was washed with H₂O, extracted with DCM, dried over anhydrous MgSO₄ and filtered. Solvent was removed under reduced pressure to give the product.

Method B. The corresponding fatty acid (1.1 equiv.) and N,N′-dicyclohexylcarbodiimide (DCC)(1.1 equiv.) were dissolved in DCM and stirred for 30 min. Subsequently DMAP (0.1 equiv.) and compound 2 (1 equiv.) were successively added and the resulting mixture stirred for 2 hours. The solution was filtered and the solvent removed under reduced pressure and the crude product redissolved in DCM (three times). The product was washed with H₂O, extracted with DCM, dried over anhydrous MgSO₄ and filtered. Solvent was removed under reduced pressure to give the product.

N-[Tris(tert-butyldimethylsilyloxymethyl)methyl]dodecamide, 3c (83%): ¹H NMR (CDCl₃) δ: 5.5 (s, 1H), 3.8 (s, 6H), 2.1-0.9 (m, 23H), 0.85 (s, 27H), 0.0 (s, 18H). ¹³C NMR (CDCl₃) δ: 171.9, 61.0, 29.6, 29.7, 26.2, 23.1, 18.6, 14.5. LRMS (ES+) 646.7 [100, (M+H)⁺].

N-[Tris(tert-butyldimethylsilyloxymethyl)methyl]pentadecamide, 3f (93%): ¹H NMR (CDCl₃) δ: 5.5 (s, 1H), 3.8 (s, 6H), 2.1-1.0 (m, 29H), 0.85 (s, 27H), 0.0 (s, 18H). ¹³C NMR (CDCl₃) δ: 171.9, 59.6, 30.9, 28.4, 28.2, 24.8, 23.7, 21.7, 17.2, 13.1. LRMS (ES+) 674.8 [100, (M+H)⁺].

N-[Tris(tert-butyldimethylsilyloxymethyl)methyl]octadecamide, 3h (92%): ¹H NMR (CDCl₃) δ: 5.5 (s, 1H), 3.8 (s, 6H), 0.9-2.1 (m, 35H), 0.85 (s, 27H), 0.0 (s, 18H). ¹³C NMR (CDCl₃) δ: 171.7, 59.6, 47.7, 32.9, 28.7, 28.4, 28.1, 25.4, 24.8, 24.7, 23.9, 21.5, 17.2, 13.1. LRMS (ES+) 730.8 [100, (M+H)⁺].

N-[Tris(tert-butyldimethylsilyloxymethyl)methyl]-9-octadecenamide, 31 (85%): ¹H NMR (CDCl₃) δ: 5.5 (s, 1H), 5.3 (t, J=5.5, 2H), 3.8 (s, 6H), 2.1-0.9 (m, 31H), 0.85 (s, 27H), 0.0 (s, 18H). ¹³C NMR (CDCl₃) δ: 171.7, 59.7, 36.7, 32.9, 30.9, 28.7, 28.5, 28.1, 26.2, 24.8, 23.9. 21.6, 17.2, 13.1. LRMS (ES+) 728.8 [100, (M+H)⁺].

### C) Synthesis of Deprotected Derivatives 4a-l

General TDMBS deprotection procedure: Tetrabutylammonium fluoride (1.5 eq) in THF was added to the compound and the mixture left stirring at room temperature for 3 hours. The solvent was removed under reduced pressure and water added. The suspension was sonicated for 1 hour and left to precipitate overnight. The product was recovered by vacuum filtration washing with diethyl ether to obtain the pure compound as a white solid (yield=55-90%).

N-[Tris(hydroxymethyl)methyl]dodecamide, 4c (69% yield): ¹H NMR (DMSO) δ: 7.3 (s, 1H), 5.0 (br s, 3H), 3.5 (s, 6H), 2.2-1.5 (m, 20H), 1.1 (t, J=6.5, 3H). ¹³C NMR (DMSO) δ: 174.2, 62.5, 61.2, 36.2, 31.7, 31.1, 29.4, 29.0, 25.7, 22.4, 14.3.

N-[Tris(hydroxymethyl)methyl]pentadecamide, 4f (81% yield): ¹H NMR (DMSO) δ: 7.5 (s, 1H), 5.3 (br s, 3H), 3.5 (s, 6H), 2.2-1.3 (m, 26H), 1.1 (t, J=6.0, 3H). ¹³C NMR (DMSO): 174.2, 62.6, 61.2, 36.2, 31.7, 29.4, 29.0, 25.7, 22.4, 14.3.

N-[Tris(hydroxymethyl)methyl]octadecamide, 4h (86% yield): ¹H NMR (DMSO) δ: 7.3 (s, 1H), 5.0 (br s, 3H), 3.5 (s, 6H), 2.2-1.5 (m, 32H), 1.1 (t, J=6.0, 3H). ¹³C NMR (DMSO) δ: 174.2, 62.5, 61.2, 36.2, 31.7, 31.1, 29.4, 29.0, 25.7, 22.4, 14.3.

N-[Tris(hydroxymethyl)methyl]-9-octadecenamide, 41 (55% yield): ¹H NMR (DMSO) δ: 7.3 (s, 1H), 5.3 (t, 2H), 5.0 (br s, 3H), 3.5 (s, 6H), 0.9-2.1 (m, 28H), 1.1 (t, J=6.6, 3H). ¹³C NMR 6: (DMSO) 174.2, 57.8, 47.9, 33.7, 25.7, 24.8, 23.4, 19.5, 13.8.

### D) Synthesis of protected compounds 5a-n

General coupling with ^{t}Boc-protected amino acids: The coupling compound (3.43 mmol, 3.3 eq) was dissolved in DCM (40 mL) and DMF (5 mL) and DCC (702 mg, 3.43 mmol, 3.3 eq) added and the mixture was stirred at room temperature for 30 min. A solution of one of 4a-l (400 mg, 1.04 mmol) was added slowly to the mixture with DMF (5 mL) and left to stir for 10 minutes. DMAP (13.0 mg, 0.1 mmol, 0.1 eq) was then added and the mixture left to stir at room temperature overnight. The solution was filtered and the solvent removed under reduced pressure, the crude product was dissolved onto silica and purified by column chromatograph using ethyl acetate/hexane mixtures. The pure product fractions were combined and the solvent evaporated under reduced pressure giving colourless oils, which crystallised under vacuum (yield 40-60%).

N-tris([4-(N-tert-butoxycarbonylamino)butanoyl]oximethyl)dodecamide, 5c (60%): ¹H NMR (CDCl₃) δ: 6.7 (s, 1H), 5.0 (br t, 3H), 4.3 (s, 6H), 3.0 (m, 6H), 2.3 (t, J=7.5, 6H), 2.0 (t, 2H, J=7.5), 1.7 (m, 6H), 1.6-1.1 (m, 45H), 0.8 (t, 3H, J=6.6). ¹³C NMR (CDCl₃) δ: 173.8, 172.2, 155.9, 78.8, 62.0, 57.7, 39.4, 36.6, 36.2, 31.6, 31.1, 30.9, 29.4, 29.3, 29.1, 29.0, 28.9, 28.1, 25.3, 25.0, 22.4, 13.8. LRMS (ES+) m/z 873.6 [100, (M+H)⁺].

N-tris([4-(N-tert-butoxycarbonylamino)butanoyl]oximethyl)pentadecamide, 5e (55%): ¹H NMR (CDCl₃) δ: 6.5 (s, 1H), 4.9 (br t, 3H), 4.4 (s, 6H), 3.0 (m, 6H), 2.3 (t, J=7.0, 6H), 2.1 (t, 2H, J=7.5), 1.7 (m, 6H), 1.6-1.1 (m, 51H), 0.8 (t, 3H, J=6.6). ¹³C NMR (CDCl₃) δ: 173.8, 172.3, 155.9, 79.1, 62.1, 57.9, 39.5, 36.7, 36.2, 31.7, 31.0, 29.5, 29.3, 29.1, 29.0, 28.9, 28.1, 25.3, 25.1, 22.5, 13.9. LRMS (ES+) m/z 915.6 [100, (M+H)⁺].

N-tris([4-(N-tert-butoxycarbonylamino)butanoyl]oximethyl)-9-octadecamide, 51 (40%): ¹H NMR (CDCl₃) δ: 6.4 (s, 1H), 4.7 (br t, 3H), 4.4 (s, 6H), 3.1 (m, 6H), 2.3 (t, J=7.5, 6H), 2.1 (t, 2H, J=7.5), 1.7 (m, 6H), 1.6-1.1 (m, 57H), 0.8 (t, 3H, J=6.6). ¹³C NMR (CDCl₃) δ: 172.9, 171.5, 155.0, 78.3, 61.3, 59.3, 57.0, 38.7, 35.9, 30.1, 29.8, 29.0, 28.7, 27.4, 24.5, 24.3, 21.3, 20.0, 13.1. (ES+) m/z 941.6 [100, (M+H)⁺].

N-tris([4-(N-tert-butoxycarbonylamino)hexanoyl]oximethyl)pentadecamide, 5m (85%): ¹H NMR (CDCl₃) δ: 6.0 (s, 1H), 4.6 (br t, 3H), 4.4 (s, 6H), 3.0 (q, J=6.5 Hz, 6H), 2.3 (t, J=7.5 Hz, 6H), 2.1 (t, J=6.0 Hz, 2H), 1.6-1.1 (m, 69H), 0.8 (t, 3H, J=7.0); LRMS (ES+) m/z 1007.6 [100, (M+Na)⁺].

N-tris([4-(N-tert-butoxycarbonylamino)hexanoyl]oximethyl)-9-octadecamide, 5n (70%): ¹H NMR (CDCl₃) δ: 5.9 (s, 1H), 5.3 (m, 2H), 4.6 (br t, 3H), 4.3 (s, 6H), 3.0 (q, J=7.0 Hz, 6H), 2.2 (t, J=7.0 Hz, 6H), 2.0 (t, J=6.0 Hz, 2H), 1.9 (br q, 4H), 1.6-1.1 (m, 67H), 0.8 (t, 3H, J=7.0); LRMS (ES+) m/z 1047.6 [100, (M+Na)⁺].

### E) Synthesis of Final Compounds 6a-n

General deprotection protocol: Trifluoroacetic acid (99%) and DCM (1:1, 10 mL) was added to 5a-l (0.1 mmol) and left to stir at room temperature for 30 mins. The solvent was evaporated under reduced pressure and the product re-dissolved in DCM and the solvent evaporated under reduced pressure (three times) to give final compounds 6a-n as sticky white solids or colourless oils (quantitative yield).

N-[tris-(4-aminobutiryloximethyl)methyl]decamide, trifluoroacetic salt, 6a. ¹H NMR (250 MHz, methanol-d) δ 4.44 (s, 6H), 2.99 (t, J=7.5 Hz, 6H), 2.52 (t, J=7.5 Hz, 6H), 2.19 (t, J=7.5 Hz, 2H), 1.95 (q, J=7.5 Hz, 6H), 1.65-1.12 (m, 14H), 0.90 (t, J=6.0 Hz, 3 H); ¹³C NMR (62.5 MHz, chloroform-d) δ 177.0, 173.4, 63.1 (CH₂), 59.1, 40.0 (CH₂), 37.5 (CH₂), 33.1 (CH₂), 31.4 (CH₂), 30.7 (CH₂), 30.5 (CH₂) 30.5 (CH₂), 30.3 (CH₂) 27.0 (CH₂), 23.8 (CH₂), 23.7 (CH₂), 14.5 (CH₃); LRMS (ES+) m/z 531.5 [100, (M+H)⁺].

N-[tris-(4-aminobutiryloximethyl)methyl]undecamide, trifluoroacetic salt, 6b. ¹H NMR (250 MHz, methanol-d) δ 4.44 (s, 6H), 2.99 (t, J=7.5 Hz, 6H), 2.51 (t, J=7.5 Hz, 6H), 2.19 (t, J=7.5 Hz, 2H), 1.95 (q, J=7.5 Hz, 6H), 1.67-1.09 (m, 16H), 0.90 (t, J=6.0 Hz, 3 H); ¹³C NMR (62.5 MHz, chloroform-d) δ 177.0, 173.4, 63.1 (CH₂), 59.2, 40.0 (CH₂), 37.5 (CH₂), 33.1 (CH₂), 31.4 (CH₂), 30.7 (CH₂), 30.7 (CH₂), 30.5 (CH₂) 30.3 (CH₂) 27.0 (CH₂), 23.7 (CH₂), 23.7 (CH₂), 14.5 (CH₃); LRMS (ES+) m/z 545.6 [100, (M+H)⁺].

N-[tris-(4-aminobutiryloximethyl)methyl]dodecamide, trifluoroacetic salt, 6c. ¹H NMR (250 MHz, methanol-d) δ 4.44 (s, 6H), 2.99 (t, J=7.5 Hz, 6H), 2.51 (t, J=7.5 Hz, 6H), 2.19 (t, J=7.5 Hz, 2H), 1.95 (q, J=7.5 Hz, 6H), 1.68-1.06 (m, 18H), 0.90 (t, J=6.0 Hz, 3 H); ¹³C NMR (62.5 MHz, chloroform-d) δ 177.0, 173.4, 63.1 (CH₂), 59.2, 40.0 (CH₂), 37.5 (CH₂), 33.1 (CH₂), 31.4 (CH₂), 30.7 (CH₂), 30.7 (CH₂), 30.5 (CH₂) 30.3 (CH₂) 27.0 (CH₂), 23.7 (CH₂), 23.7 (CH₂), 14.5 (CH₃); LRMS (ES+) m/z 559.6 [100, (M+H)⁺].

N-[tris-(4-aminobutiryloximethyl)methyl]tridecamide, trifluoroacetic salt, 6d. ¹H NMR (250 MHz, methanol-d) δ 4.44 (s, 6H), 2.99 (t, J=7.5 Hz, 6H), 2.51 (t, J=7.5 Hz, 6H), 2.19 (t, J=7.5 Hz, 2H), 1.95 (q, J=7.5 Hz, 6H), 1.66-1.00 (m, 20H), 0.89 (t, J=6.0 Hz, 3 H); ¹³C NMR (62.5 MHz, chloroform-d) δ 177.0, 173.4, 63.1 (CH₂), 59.2, 40.0 (CH₂), 37.5 (CH₂), 33.1 (CH₂), 31.5 (CH₂), 30.8 (CH₂), 30.7 (CH₂), 30.5 (CH₂) 30.3 (CH₂) 27.0 (CH₂), 23.7 (CH₂), 23.7 (CH₂), 14.5 (CH₃); LRMS (ES+) m/z 545.6 [100, (M+H)⁺].

N-[tris-(4-aminobutiryloximethyl)methyl]tetradecamide, trifluoroacetic salt, 6e. ¹H NMR (250 MHz, methanol-d) δ 4.44 (s, 6H), 2.99 (t, J=7.5 Hz, 6H), 2.51 (t, J=7.5 Hz, 6 H), 2.19 (t, J=7.5 Hz, 2H), 1.95 (q, J=7.5 Hz, 6H), 1.67-1.02 (m, 22H), 0.90 (t, J=6.5 Hz, 3H); ¹³C NMR (62.5 MHz, chloroform-d) δ 177.0, 173.4, 63.1 (CH₂), 59.1, 40.0 (CH₂), 37.5 (CH₂), 33.1 (CH₂), 31.4 (CH₂), 30.8 (CH₂), 30.7 (CH₂), 30.5 (CH₂), 30.3 (CH₂), 27.0 (CH₂), 23.8 (CH₂), 23.7 (CH₂), 14.5 (CH₃); LRMS (ES+) m/z 587.7 [100, (M+H)⁺].

N-[tris-(4-aminobutiryloximethyl)methyl]pentadecamide, trifluoroacetic salt, 6f. ¹H NMR (250 MHz, methanol-d) δ 4.44 (s, 6H), 2.99 (t, J=7.5 Hz, 6H), 2.51 (t, J=7.5 Hz, 6 H), 2.19 (t, J=7.5 Hz, 2H), 1.95 (q, J=7.5 Hz, 6H), 1.69-1.06 (m, 24H), 0.90 (t, J=6.0 Hz, 3H); ¹³C NMR (62.5 MHz, chloroform-d) δ 177.0, 173.4, 63.1 (CH₂), 59.2, 40.0 (CH₂), 37.5 (CH₂), 33.1 (CH₂), 31.4 (CH₂), 30.9 (CH₂), 30.7 (CH₂), 30.5 (CH₂) 30.3 (CH₂) 27.0 (CH₂), 23.7 (CH₂), 23.7 (CH₂), 14.5 (CH₃); LRMS (ES+) m/z 601.7 [100, (M+H)⁺].

N-[tris-(4-aminobutiryloximethyl)methyl]hexadecamide, trifluoroacetic salt, 6g. ¹H NMR (250 MHz, methanol-δ) δ 4.44 (s, 6H), 2.99 (t, J=7.5 Hz, 6H), 2.51 (t, J=7.5 Hz, 6 H), 2.19 (t, J=7.5 Hz, 2H), 1.95 (q, J=7.5 Hz, 6H), 1.70-1.03 (m, 26H), 0.90 (t, J=6.0 Hz, 3H); ¹³C NMR (62.5 MHz, chloroform-d) δ 177.0, 173.4, 63.1 (CH₂), 59.1, 40.0 (CH₂), 37.5 (CH₂), 33.1 (CH₂), 31.4 (CH₂), 30.8 (CH₂), 30.7 (CH₂), 30.5 (CH₂) 30.3 (CH₂) 27.0 (CH₂), 23.8 (CH₂), 23.7 (CH₂), 14.5 (CH₃); LRMS (ES+) m/z 615.7 [100, (M+H)⁺].

N-[tris-(4-aminobutiryloximethyl)methyl]octadecamide, trifluoroacetic salt, 6h. ¹H NMR (250 MHz, methanol-δ) δ 4.44 (s, 6H), 2.99 (t, J=7.5 Hz, 6H), 2.51 (t, J=7.5 Hz, 6 H), 2.19 (t, J=7.5 Hz, 2H), 1.95 (q, J=7.5 Hz, 6H), 1.67-1.01 (m, 30H), 0.90 (t, J=6.0 Hz, 3H); ¹³C NMR (62.5 MHz, chloroform-d) δ 177.0, 173.4, 63.1 (CH₂), 59.1, 40.0 (CH₂), 37.4 (CH₂), 33.1 (CH₂), 31.4 (CH₂), 30.8 (CH₂), 30.7 (CH₂), 30.5 (CH₂) 30.3 (CH₂) 27.0 (CH₂), 23.7 (CH₂), 23.7 (CH₂), 14.5 (CH₃); LRMS (ES+) m/z 643.7 [100, (M+H)⁺].

N-[tris-(4-aminobutiryloximethyl)methyl]-9-octadecenamide, trifluoroacetic salt, 6i. ¹H NMR (250 MHz, methanol-d) δ 5.41-5.28 (m, 2H) 4.44 (s, 6H), 2.99 (t, J=7.5 Hz, 6H), 2.51 (t, J=7.0 Hz, 6H), 2.19 (t, J=7.5 Hz, 2H), 1.95 (q, J=7.5 Hz, 6H), 1.71-0.92 (m, 26 H), 0.90 (t, J=6.5 Hz, 3H); ¹³C NMR (62.5 MHz, chloroform-d) δ 177.0, 173.4, 130.9, (CH), 130.8, (CH), 63.1 (CH₂), 59.2, 40.0 (CH₂), 37.4 (CH₂), 33.1 (CH₂), 31.4 (CH₂), 30.9 (CH₂), 30.6 (CH₂), 30.4 (CH₂) 30.4 (CH₂) 30.4 (CH₂) 30.3 (CH₂), 28.2 (CH₂) 27.0 (CH₂), 23.7 (CH₂), 23.7 (CH₂), 14.5 (CH₃); LRMS (ES+) m/z 641.7 [100, (M+H)⁺].

N-[tris-(4-aminobutiryloximethyl)methyl]eicosanamide, trifluoroacetic salt, 6j. ¹H NMR (250 MHz, methanol-d) δ 4.44 (s, 6H), 2.99 (t, J=7.5 Hz, 6H), 2.51 (t, J=7.0 Hz, 6 H), 2.19 (t, J=7.5 Hz, 2H), 1.95 (q, J=7.5 Hz, 6H), 1.65-0.99 (m, 34H), 0.90 (t, J=6.5 Hz, 3H); ¹³C NMR (62.5 MHz, chloroform-d) δ 177.0, 173.4, 63.1 (CH₂), 59.2, 40.0 (CH₂), 37.5 (CH₂), 33.1 (CH₂), 31.4 (CH₂), 30.8 (CH₂), 30.5 (CH₂), 30.3 (CH₂), 27.0 (CH₂), 23.8 (CH₂), 23.7 (CH₂), 14.5 (CH₃); LRMS (ES+) m/z 671.8 [100, (M+H)⁺].

N-[tris-(4-aminobutiryloximethyl)methyl]tetraeicosanamide, trifluoroacetic salt, 6k. ¹H NMR (250 MHz, methanol-d) δ 4.44 (s, 6H), 2.99 (t, J=7.5 Hz, 6H), 2.51 (t, J=7.5 Hz, 6 H), 2.19 (t, J=7.5 Hz, 2H), 1.95 (q, J=7.5 Hz; 6H), 1.69-1.06 (m, 42H), 0.90 (t, J=6.5 Hz, 3H); ¹³C NMR (62.5 MHz, chloroform-d) δ 177.0, 173.8, 63.5 (CH₂), 59.5, 40.4 (CH₂), 37.9 (CH₂), 33.5 (CH₂), 31.8 (CH₂), 31.2 (CH₂), 30.9 (CH₂), 30.7 (CH₂), 27.4 (CH₂), 24.2 (CH₂), 24.1 (CH₂), 14.9 (CH₃); LRMS (ES+) m/z 727.7 [100, (M+H)⁺].

O-(Cholest-5-en-3-yl)-N-[tris-(4-aminobutiryloxymethyl)methyl]carbamate, trifluoroacetic salt, 6l. ¹H NMR (250 MHz, methanol-d) δ 5.47 (s, 1H), 4.44 (s, 6H), 2.99 (t, J=7.5 Hz, 6H), 2.51 (t, J=7.5 Hz, 6H), 1.95 (q, J=7.5 Hz, 6H), 1.70-0.69 (m, 45H); ¹³C NMR (62.5 MHz, chloroform-d) δ 173.4, 157.0, 141.1, 123.6 (CH), 63.3 (CH₂), 58.4, 58.2, 57.6, 54.8, 51.6, 43.5 (CH₂), 41.1, 40.8, 40.7, 40.2, 40.1, 40.0, 40.0, 38.3, 37.8 (CH₂), 37.4, 37.1, 33.2 (CH₂), 33.1, 31.4 (CH₂), 29.3, 29.2 (CH₂), 25.3, 25.0, 23.6, 23.3, 23.0, 22.2, 19.8, 19.3, 12.4; LRMS (ES+) m/z 789.8 [100, (M+H)⁺].

N-[tris-(4-aminohexanoyloxymethyl)methyl]pentadecamide, trifluoroacetic salt, 6m. ¹H NMR (250 MHz, methanol-d) δ 4.40 (s, 6H), 2.93 (t, J=7.5 Hz, 6H), 2.39 (t, J=7.5 Hz, 6H), 2.18 (t, J=6.0 Hz, 2H), 1.77-1.17 (m, 42H), 0.90 (t, J=7.0 Hz, 3H); LRMS (ES+) m/z 685.6 [100, (M+H)⁺].

N-[tris-(4-aminohexanoyloxymethyl)methyl]-9-octadecenamide, trifluoroacetic salt, 6n. ¹H NMR (250 MHz, methanol-d) δ 5.22 (m, 2H), 4.42 (s, 6H), 2.96 (t, J=7.5 Hz, 6H), 2.75 (m, 6H), 2.18 (t, J=6.5 Hz, 2H), 1.95 (br q, 4H), 1.79-1.18 (m, 40H), 0.90 (t, J=7.0 Hz, 3H); LRMS (ES+) m/z 725.6 [100, (M+H)⁴].

2.2. Compounds with General Structure:

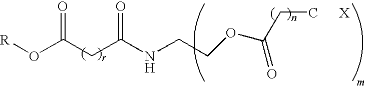

In particular compounds with this general structure and having:
R—O— a fatty alkyloxy chain such as decyloxy, undecyloxy, dodecyloxy, tridecyloxy, tetradecyloxy, pentadecyloxy, hexadecyloxy, octadecyloxy, 9-octadecenyloxy (oleyloxy), eicosyloxy, or tetraeicosyloxy.
r=2, 3, or 4
n=1, 2, 3, 4 or 5
m=2 or 3

### C=—NH₂ or —NH—C(═NH)NH₂

### X=CF₃—C(═O)OH or HCl

and

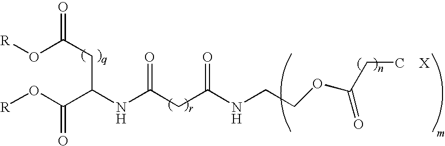

In particular compounds with this general structure and having:
R—O— a fatty alkyloxy chain such as decyloxy, undecyloxy, dodecyloxy, tridecyloxy, tetradecyloxy, pentadecyloxy, hexadecyloxy, octadecyloxy, 9-octadecenyloxy, eicosyloxy, or tetraeicosyloxy.
q=1 or 2
r=2, 3, or 4
n=1, 2, 3, 4 or 5
m=2 or 3

### C=—NH₂ or —NH—C(═NH)NH₂

### X=CF₃—C(═O)OH or HCl

### 2.2.1 Synthesis of Intermediate 11

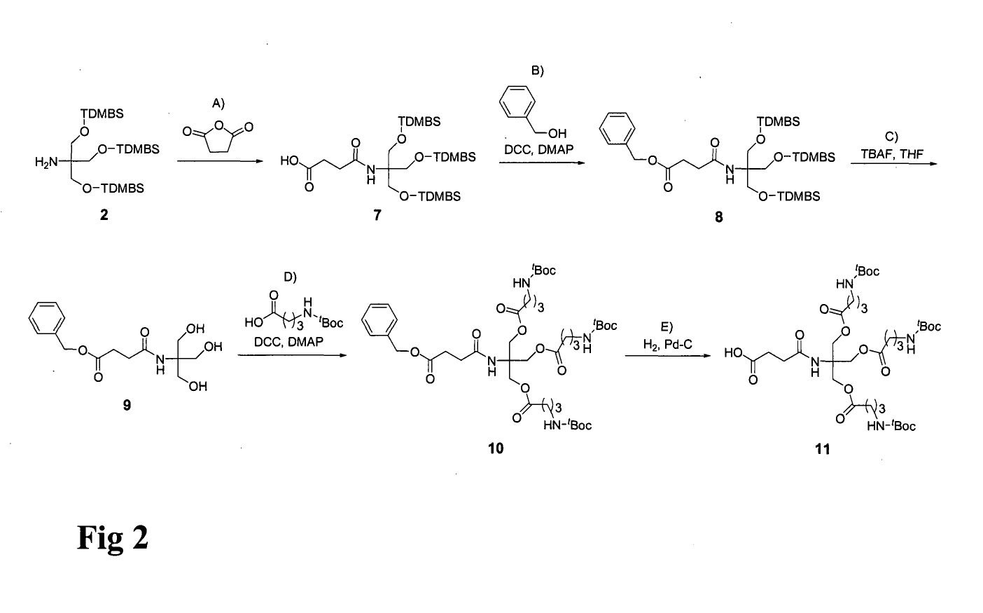

Synthesis of intermediate 11 of the invention was achieved by following the synthetic strategy outlined in the scheme shown in FIG. 2.

### A) Synthesis of [tris(tert-butyldimethylsilyloxymethyl)methyl]amidosuccinic acid, 7

Tris(tert-butyldimethylsilyloxymethyl)aminomethane 2 (5.75 g, 12.4 mmol) and succinic anhydride (1.86 g, 18.60 mmol) were dissolved in DCM (10 ml). DMAP (0.15 g, 1.24 mmol, 0.1 eq) was then added and the mixture stirred at room temperature overnight. The product was extracted using a 5% NaHCO₃ solution, then acidified with 2N HCl and extracted with DCM. The organic phase was dried over anhydrous MgSO₄ and filtered. Solvent was removed in vacuo to give a colourless oil. (7.00 g, 12:4 mmol, quantitative yield). ¹H NMR (CDCl₃) δ: 7.2 (s, 1H), 5.8 (s, 1H), 3.8 (s, 6H), 2.6 (t, J=6.0 Hz, 2H), 2.4 (t, J=6.0 Hz, 2H), 0.8 (s, 27H), 0.0 (s, 18H); LRMS (ES+) m/z 565.3 [100, (M+H⁺)].

### B) Synthesis of benzyl [tris(tert-butyldimethylsilyloxymethyl)methyl]amidosuccinate, 8

Compound 7 (7.00 g, 12.4 mmol), DCC (3.84 g, 18.6 mmol) and DMAP (0.15 g, 1.24 mmol) were dissolved in DCM (20 mL). Benzyl alcohol (1.48 g, 13.6 mmol) was then added and the mixture stirred at room temperature for 2 hours. The solution was filtered (to remove DCC) and solvent removed in vacuo. The crude product was re-dissolved in DCM, washed with water, and dried over anhydrous MgSO₄. Solvent was removed in vacuo to give a colourless oil (7.00 g, 10.7 mmol, 86%). ¹H NMR (CDCl₃) δ 7.3 (m, 5H), 5.6 (s, 1H), 5.1 (s, 2H), 3.8 (s, 6H), 2.6 (t, J=6.0 Hz, 2H), 2.4 (t, J=6.0 Hz, 2H), 0.8 (s, 27H), 0.0 (s, 18H); LRMS (ES+) m/z 654.4 [100, (M+H)⁺].

### C) Synthesis of benzyl tris(hydroxymethyl)amidosuccinate, 9

Compound 8 was added to a solution of CH₃COOH:THF:H₂O (3:5:1) and the mixture was stirred at room temperature overnight. Solvents were removed under reduced pressure to give a sticky, white solid. ¹H NMR (CDCl₃) δ 7.3 (m, 5H), 7.1 (s, 1H), 5.0 (s, 2H), 3.6 (s, 6H), 2.6 (t, J=6.0 Hz, 2H), 2.4 (t, J=6.0 Hz, 2H); LRMS (ES+) m/z 334.0 [100, (M+Na⁺)].

### D) Synthesis of benzyl [tris(4-[N-tert-butoxycarbonylamino]butanoyloxymethyl)methyl]amidosuccinate, 10

N-^{t}Boc-GABA (431.1 mg, 2.12 mmol), DMAP (8.4 mg, 0.06 mmol), EDC (407 mg, 2.12 mmol) and compound 9 (200 mg, 0.64 mmol) were dissolved in DCM:DMF (1:1, 2 mL) and microwave irradiated for 20 minutes at 60° C. The product was washed with water, extracted with DCM, dried over anhydrous MgSO₄ and purified by flash chromatography using ethyl acetate/hexane mixtures to afford a clear, colourless oil (443 mg, 0.5 μmol, 80%). ¹H NMR (CDCl₃) δ 7.3 (m, 5H), 6.8 (s, 1H), 5.0 (s, 2H), 4.7 (br s, 3H) 4.4 (s, 6H), 3.1 (m, 6H), 2.6 (t, J=6.0 Hz, 2H), 2.4 (t, J=6.0 Hz, 2H), 2.3 (m, 2H), 1.6 (m, 6H), 1.4 (s, 27H); LRMS (ES+) m/z 889.3 [100, (M+Na⁺)].

### E) Synthesis of [tris(4-[N-tert-butoxycarbonylamino]butanoyloxymethyl)methyl]amidosuccinic acid, 11

Compound 10 (443.5 mg, 0.51 mmol) was dissolved in ethanol (20 mL) and Pd—C (45 mg) was added and the system closed. A H₂ balloon was then connected to the system and the mixture was stirred at room temperature for 2 hours. The product was filtered through celite and solvents removed in vacuo to give a colourless oil (350 mg, 0.45 mmol, 90%). ¹H NMR (CDCl₃) δ 7.9 (s, 1H), 6.8 (s, 1H), 4.7 (br s, 3H), 4.4 (s, 6H), 3.1 (m, 6H), 2.6 (t, J=6.0 Hz, 2H), 2.4-2.1 (m, 8H), 1.6 (m, 6H), 1.4 (s, 27H); LRMS (ES+) m/z 799.2 [100, (M+Na⁺)].

### 2.2.1 Compounds 13a,b and 15a,b

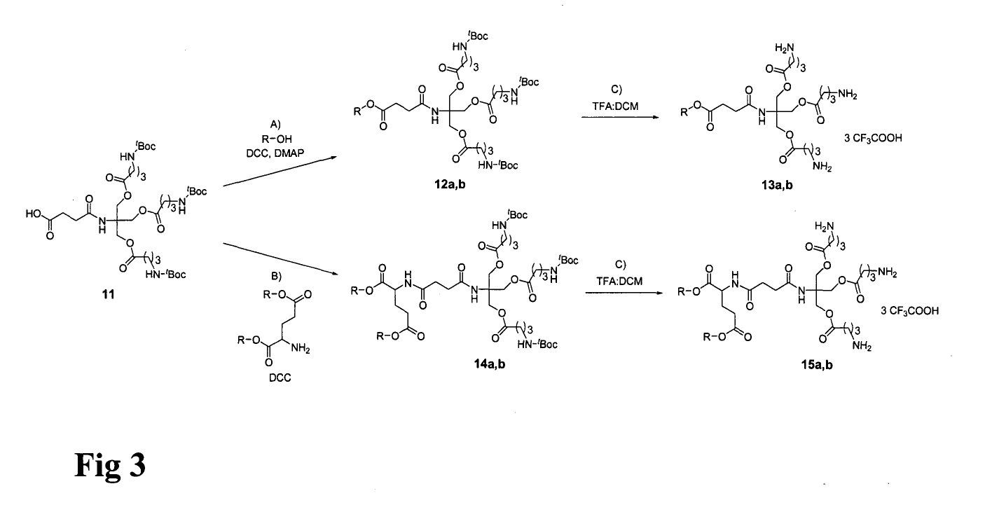

Synthesis of 13a,b and 15a,b the invention was achieved by following the synthetic strategy outlined in the scheme shown in FIG. 3. The groups R—O— are as follows for each compound: 13a pentadecyloxy, 13b 9-octadecenyloxy, 15a pentadecyloxy, 15b 9-octadecenyloxy.

### A) Synthesis of ^{t}Boc-protected derivatives 12a,b

The corresponding fatty alcohol (1.1 equiv.) and DCC (1.1 equiv.) were dissolved in DCM and stirred for 30 min. Then DMAP (0.1 equiv.) was added to the solution and, subsequently, compound 11 (1 equiv.) was added and the resulting mixture stirred for 2 hours. The solution was filtered and the solvent removed under reduced pressure and the crude product redissolved in DCM (three times). The product was washed with H₂O, extracted with DCM, dried over anhydrous MgSO₄ and purified by flash chromatography using ethyl acetate/hexane mixtures to give the products as colourless oils.

Pentadecyl [tris(4-[N-tert-butoxycarbonylamino]butanoyloxymethyl)methyl]amidosuccinate, 12a (76%). ¹H NMR (250 MHz, methanol-d) δ 6.62 (br s, 1H), 4.75 (br t, 3H), 4.37 (s, 6H), 3.98 (t, J=7.0 Hz, 2H), 3.09 (q, J=7.0 Hz, 6H), 2.54 (t, J=6.0 Hz, 2H), 2.40 (t, J=6.0 Hz, 2H), 2.32 (t, J=7.0 Hz, 6H), 1.73 (m, 6H), 1.61-1.03 (m, 53H), 0.81 (t, J=7.0 Hz, 3H); LRMS (ES+) m/z 1009.6 [100, (M+Na)^{4].}

9-octadecenyl [tris(4-[N-tert-butoxycarbonylamino]butanoyloxymethyl)methyl]amidosuccinate, 12b (82%). ¹H NMR (250 MHz, methanol-d) δ 6.60 (br s, 1H), 5.27 (m, 2H), 4.74 (br t, 3H), 4.37 (s, 6H), 3.98 (t, J=7.0 Hz, 2H), 3.09 (br q, J=6.5 Hz, 6H), 2.54 (t, J=6.0 Hz, 2H), 2.40 (t, J=6.0 Hz, 2H), 2.31 (t, J=7.0 Hz, 6H), 1.95 (m, 4H), 1.73 (m, 6H), 1.61-1.03 (m, 53H), 0.81 (t, J=6.5 Hz, 3H); LRMS (ES+) m/z 1009.6 [100, (M+Na)⁺].

### B) Synthesis of ^{t}Boc-protected derivatives 14a,b

The O,O′-dialkyl glutamate (obtained as described in Obata et al. Bioconjugate Chem., 19, 1055-1063, 2008) (1.1 equiv.) and DCC (1.1 equiv.) were dissolved in DCM and stirred for 30 min. Subsequently, compound 11 (1 equiv.) was added and the resulting mixture stirred for 2 hours. The solution was filtered and the solvent removed under reduced pressure and the crude product redissolved in DCM (three times). The product was washed with H₂O, extracted with DCM, dried over anhydrous MgSO₄ and purified by flash chromatography using ethyl acetate/hexane mixtures to give the products as colourless oils (56-81%).

1,3-bis(pentadecyloxycarbonyl)-propyl [tris(4-[N-tert-butoxycarbonylamino]butanoyloxymethyl)methyl]amidosuccinamide, 14a (66%). ¹H NMR (250 MHz, methanol-d) δ 6.81 (br s, 1H), 4.83 (br t, 3H), 4.52-4.25 (m, 9H), 4.05-3.95 (m, 6H), 3.08 (br q, J=6.5 Hz, 6H), 2.31 (m, 10H), 1.73 (m, 6H), 1.60-1.01 (m, 79H), 0.81 (t, J=6.5 Hz, 6H); LRMS (ES+) m/z 1349.0 [100, (M+Na)⁺].

1,3-bis(9-octadecenyloxycarbonyl)-propyl [tris(4-[N-tert-butoxycarbonylamino]butanoyloxymethyl)methyl]amidosuccinamide, 14b (72%). ¹H NMR (250 MHz, methanol-d) δ 6.82 (br s, 1H), 5.26 (m, 4H), 4.82 (br t, 3H), 4.54-4.25 (m, 9H), 4.05-3.92 (m, 6H), 3.08 (br q, J=6.5 Hz, 6H), 2.31 (m, 10H), 1.98 (m, 8H), 1.72 (m, 6H), 1.61-1.01 (m, 79H), 0.81 (t, J=6.5 Hz, 6H); LRMS (ES+) m/z 1429.0 [100, (M+Na)⁺].

### C) Synthesis of final compounds 13a,b and 15a,b

General deprotection protocol: Trifluoroacetic acid (99%) and DCM (1:1, 10 mL) was added to 12a,b or 14a,b (0.1 mmol) and left to stir at room temperature for 30 min. The solvent was evaporated under reduced pressure and the product re-dissolved in DCM and the solvent evaporated under reduced pressure (three times) to give final compounds as colourless oils (quantitative yield).

Pentadecyl [tris(4-aminobutanoyloxymethyl)methyl]amidosuccinate, trifluoroacetic salt, 13a. ¹H NMR (250 MHz, methanol-d) δ 4.45 (s, 6H), 4.08 (t, J=6.5 Hz, 2H), 3.06 (br t, J=7.0 Hz, 6H), 2.54 (m, 10H), 1.98 (m, 6H), 1.67 (m, 2H), 1.48-1.20 (m, 26H), 0.91 (t, J=7.0 Hz, 3H); LRMS (ES+) m/z 687.6 [100, (M+H)⁺].

9-octadecenyl [tris(4-aminobutanoyloxymethyl)methyl]amidosuccinate, trifluoroacetic salt, 13b. ¹H NMR (250 MHz, methanol-d) δ 5.38 (m, 2H), 4.46 (s, 6H), 4.09 (t, J=6.5 Hz, 2H), 3.03 (br t, J=7.5 Hz, 6H), 2.56 (m, 10H), 2.11-1.92 (m, 10H), 1.66 (m, 2H), 1.45-1.22 (m, 24H), 0.93 (t, J=7.0 Hz, 3H); LRMS (ES+) m/z 727.6 [100, (M+H)⁺].

1,3-bis(pentadecyloxycarbonyl)-propyl [tris(4-aminobutanoyloxymethyl)methyl]amidosuccinamide, trifluoroacetic salt, 15a. NMR (250 MHz, methanol-d) δ 4.45 (s, 6H), 4.21-4.03 (m, 4H), 3.01 (t, J=7.0 Hz, 6H), 2.59-2.42 (m, 10H), 1.98 (m, 6H), 1.65 (m, 4H), 1.50-1.11 (m, 48H), 0.91 (t, J=7.0 Hz, 6H); LRMS (ES+) m/z 1027.0 [100, (M+H)⁺].

1,3-bis(9-octadecenyloxycarbonyl)-propyl [4-aminobutanoyloxymethyl)methyl]amidosuccinamide, trifluoroacetic salt, 15b. ¹H NMR (250 MHz, methanol-d) δ 5.31 (m, 4H), 4.45 (s, 6H), 4.21-4.03 (m, 4H), 3.01 (t, J=7.0 Hz, 6H), 2.59-2.42 (m, 10H), 2.08-1.88 (m, 14H), 1.65 (m, 4H), 1.50-1.1.1 (m, 48H), 0.91 (t, J=7.0 Hz, 6H); LRMS (ES+) m/z 1027.0 [100, (M+H)⁺].

2.3. Compounds with General Structure:

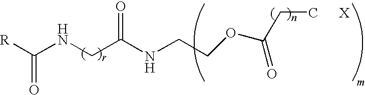

In particular compounds with this general structure and having:
R—C(═O)— a fatty acyl chain such as decanoyl, undecanoyl, dodecanoyl, tridecanoyl, tetradecanoyl, pentadecanoyl, hexadecanoyl, octadecanoyl, 9-octadecenoyl (oleyl), eicosanoyl, or tetraeicosanoyl.
r=1, 2, 3, 4 or 5
n=1, 2, 3, 4 or 5
m=2 or 3

### C=—NH₂ or —NH—C(═NH)NH₂

### X=CF₃—C(═O)OH or HCl

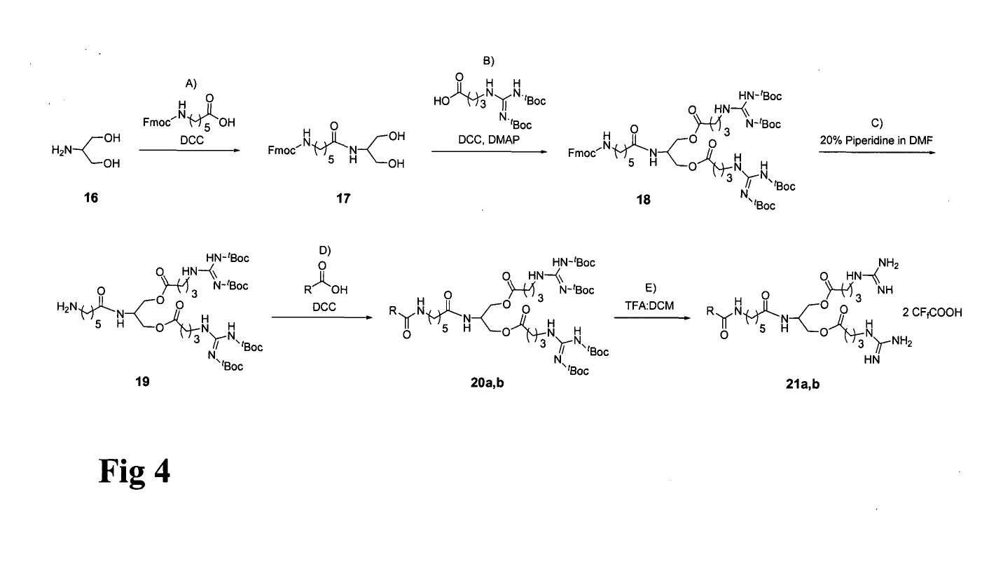

2.3.1 Synthesis of compounds 21a,b
Synthesis of cationic lipids 21a,b the invention was achieved by following the synthetic strategy outlined in the scheme shown in FIG. 4. For these compounds the groups R—C(═O)— are as follows: 21a pentadecyloxy, 21b 9-octadecenyloxy.

### A) Synthesis of N-[bis(hydroxymethyl)methyl]-6-[N′-(fluorenyl-9-methoxycarbonyl)-amino]hexanamide, 17

Fmoc-aminohexanoic acid (12.4 mmol) and DCC (13 mmol) were dissolved in DCM (20 mL) and stirred for 30 min. Subsequently, serinol 16 (13.6 mmol) was added and the mixture stirred at room temperature for 2 hours. The solution was filtered (to remove DCU) and solvent removed in vacuo. The crude product was re-dissolved in DCM, washed with water, and dried over anhydrous MgSO₄, and purified by flash chromatography using ethyl acetate/hexane mixtures to give a white solid (86%). LRMS (ES+) m/z 427.2 [100, (M+H⁺)].

### B) Synthesis of N-[bis(4-[N,N′-bis(tert-butoxycarbonyl)guanidino]butanoyloxymethyl)methyl]-6-[N″-(fluorenyl-9-methoxycarbonyl)-amino]hexanamide, 18

4-(N,N′-bis(tert-butoxycarbonyl)guanidino)butyric acid (2.2 mmol), DMAP (0.06 mmol), EDC (2.2 mmol) and compound 17 (1 mmol) were dissolved in DCM:DMF (1:1, 2 mL) and microwave irradiated for 20 minutes at 60° C. The product was washed with water, extracted with DCM, dried over anhydrous MgSO₄ and purified by flash chromatography using ethyl acetate/hexane mixtures to afford a colourless oil (80%). LRMS (ES+) m/z 1103.6 [100, (M+Na⁺)].

### C) Synthesis of N-[bis(4-[N′,N″-bis(tert-butoxycarbonyl)guanidino]butanoyloxy methylimethyl]-6-aminohexanamide, 19

Compound 18 (1 mmol) was dissolved in 20% piperidine in DMF (10 mL) and left to stir at room temperature for 1 h. The solvent was evaporated under reduced pressure and the product purified by flash chromatography using ethyl acetate/hexane mixtures to afford a white solid (80%). LRMS (ES+) m/z 859.5 [100, (M+H⁺)].

### D) Synthesis of ^{t}Boc-protected derivatives 20a,b

The corresponding fatty acid (1.1 equiv.) and DCC (1.1 equiv.) were dissolved in DCM and stirred for 30 min. Subsequently, compound 19 (1 equiv.) was added and the resulting mixture stirred for 2 hours. The solution was filtered and the solvent removed under reduced pressure and the crude product redissolved in DCM (three times). The product was washed with H₂O, extracted with DCM, dried over anhydrous MgSO₄ and purified by flash chromatography using ethyl acetate/hexane mixtures to give the products as colourless oils.

N-[bis(4-[N′,N″-bis(tert-butoxycarbonyl)guanidino]butanoyloxymethyl)methyl]-6-[N′″-(pentadecanoyl)amino]hexanamide, 20a. LRMS (ES+) m/z 1105.7 [100, (M+Na⁺)].

N-[bis(4-[N′,N″-bis(tert-butoxycarbonyl)guanidino]butanoyloxymethylimethyl]-6-. [N′″-(9-octadecenoyl)amino]hexanamide, 20b. LRMS (ES+) m/z 1145.8 [100, (M+Na⁺)].

### E) Synthesis of final compounds 21a,b

General deprotection protocol: Trifluoroacetic acid (99%) and DCM (1:1, 10 mL) was added to 20a,b (0.1 mmol) and left to stir at room temperature for 30 min. The solvent was evaporated under reduced pressure and the product re-dissolved in DCM and the solvent evaporated under reduced pressure (three times) to give final compounds as colourless oils (quantitative yield).

N-[bis(4-aminobutanoyloxymethyl)methyl]-6-[N′-(pentadecanoyl)amino]hexanamide, trifluoroacetic salt, 21a. LRMS (ES+) m/z 683.5 [100, (M+H⁺)].

N-[bis(4-aminobutanoyloxymethyl)methyl]-6-[N′-(9-octadecenoyl)amino]hexanamide, trifluoroacetic salt, 21b. LRMS (ES+) m/z 723.6 [100, (M+H⁺)].

### 3. Evaluation of Transfection Properties

### General Lipoplex Preparation Protocol

The corresponding cationic lipid 6a-l (1 mM in methanol) with or without DOPE (1 mM in methanol) were mixed in different proportions and the organic solvent removed by evaporation in an oven (37° C.) overnight. The resultant thin films were then hydrated with PBS. The solutions were vortexed (10-20 s) and incubated at ambient temperature for 30 min. Plasmid DNA or siRNA solution (0.04 mg/mL in PBS or another isotonic solution) was subsequently added and the solution vortexed (10-20 s). The lipoplexes were incubated at room temperature for 30 min before being used.

Transfection of pEGFP-C1 into HeLa Cell Line

Human HeLa cells were grown in RPMI supplemented with 4 mM glutamine, 10% FCS and 100 units/ml penicillin/streptomycin (RPMI-CM) until 80% confluence. Cells were then suspended using trypsin/EDTA and counted. 2×10⁴ cells in 150 μL of RPMI-CM per well were seeded in 96 well plates and incubated overnight. The day afterwards, the different lipoplex formulations were added. Each experiment was performed in quadruplicate, using Effectene® Transfection Reagent (Qiagen) and Lipofectamine™ 2000 (Invitrogen) as positive control and untreated cells as negative control.

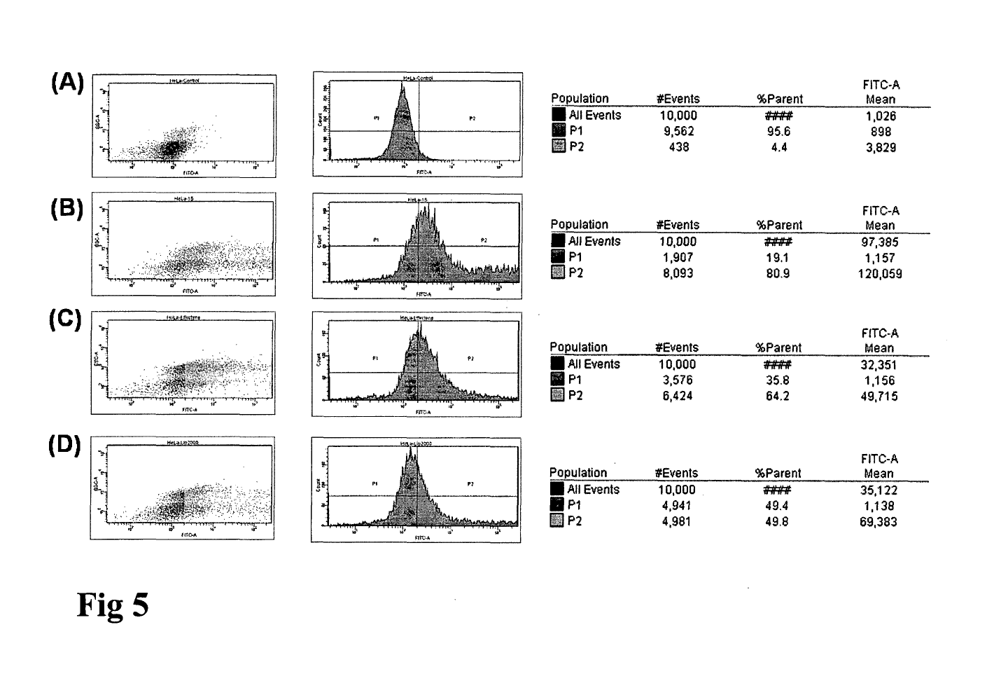

After incubation for two days at 37° C. and 5% CO₂ (the change of media after transfection was not necessary), the green fluorescent protein (GFP) expression, consequence of transfecting the pEGFP-C1, was evidenced using a fluorescent microscope (Leica) and measured by flow cytometry. For the flow cytometry analysis, cells were washed twice with PBS, detached with trypsin/EDTA, harvested with 2% ferum bovine serum (FBS) in PBS, centrifuged and resuspended with 2% FBS in PBS and analyzed using a BD FACSaria flow cytometer. Transfection efficiency was measured as percentage of transfected cells and total mean fluorescence. As shown in FIG. 5, pentadecanoyl derivative 6f (N/P ratio 12, 1:2 mol mixture with DOPE: transfected HeLa cells much better than the two positive controls (Lip2000 and Effectene® Transfection Reagent). FIG. 5 shows flow cytometry analysis of HeLa cells 48 h after transfection with a reporter plasmid: (A) shows data from an untransfected cell control; B) shows data from a compound 6f of the invention, N/P 12, 1:2 mol mixture with DOPE; (C) shows data obtained with Effectene®Transfection Reagent; and (D) shows data obtained with Lipofectamine™ 2000. The number of cells analyzed per sample was 10,000. The first column of graphs shows cell size in the y-axis and fluorescence intensity in the x-axis. Each point represents a cell. The second column of graphs represents cell number in the y-axis and fluorescence intensity in the x-axis. All the graphs have been divided into two populations of cells according with the fluorescence intensity in order to give a reference for the transfection analysis: in the untransfected cell control P1 comprises 95% of the total number of cells analyzed; when cells are transfected with compound 6f P1 comprises 19.1% of the total number of cells analyzed; within the cells transfected with Effectene® Transfection Reagent P1 comprises 35.8% of the total number of cells analyzed; when cells transfected with Lipofectamine™ 2000, P1 comprises 49.8% of the total number of cells analyzed. FITC-A Mean is the mean fluorescence (arbitrary units) calculated for each population. The mean fluorescence calculated for P2 of the cells transfected with compound 6f is 120,059, which is, by far, the best result of (A)-(D).

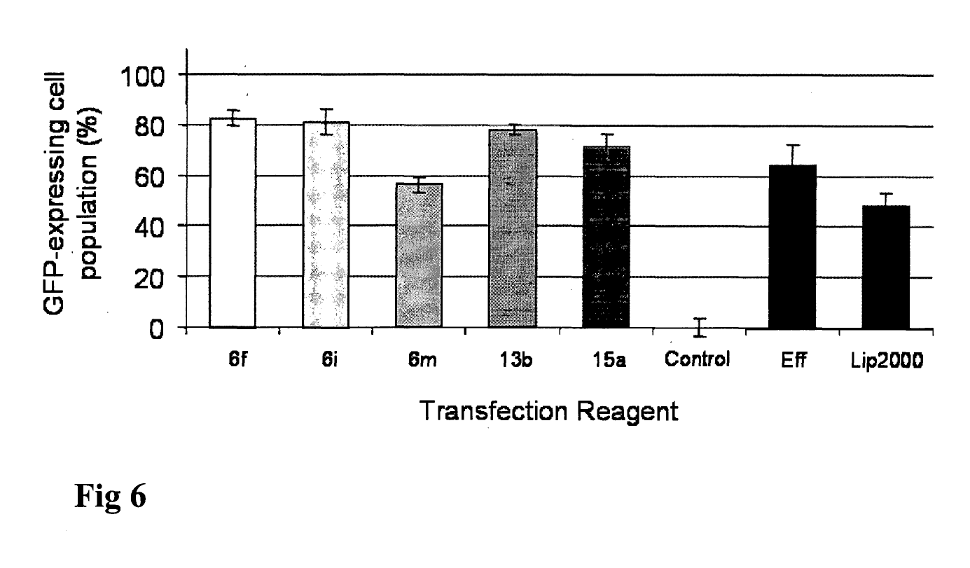

FIG. 6 shows percentage of transfected cells (calculated by flow cytometry analysis of cell fluorescence as explained before) of HeLa cells 48 hours after transfection with a GFP-reporter plasmid. Compounds 6f, 6i, 6m, 13b, and 15a of the invention were assayed and compared with Lipofectamine™ 2000 and Effectene® Transfection Reagent. FIG. 6 shows that compounds of the invention obtained GFP-expressing cell populations over 50%, giving higher transfection than Lipofectamine™ 2000. Specifically 6f and 6i produced a percentage of fluorescent cells over 80%, and 13b obtained 79%.

Transfection of pEGFP-C1 into HEK293T and B16F10 Cell Lines

Compounds 6f and 6i were successfully assayed for the transfection of pEGFP-C1 in human embryonic kidney (HEK) 293T cell line (62% and 54% of GFP-expressing cells calculated via flow cytometry, respectively) and B16F10 mouse melanoma cells (43% and 61%, respectively), giving comparable or higher transfection than Lipofectamine™ 2000 (68% in HEK293T and 44% in B16F10).

### Cell Viability Assay of Transfected HeLa Cells

Twenty-four hours after the addition of transfection agents, HeLa cell viability was measured using an MTT cell proliferation assay (LGC Promochem, Middlesex, UK), which was performed according to the manufacturer's instructions. Absorbance was read at 570 nm.

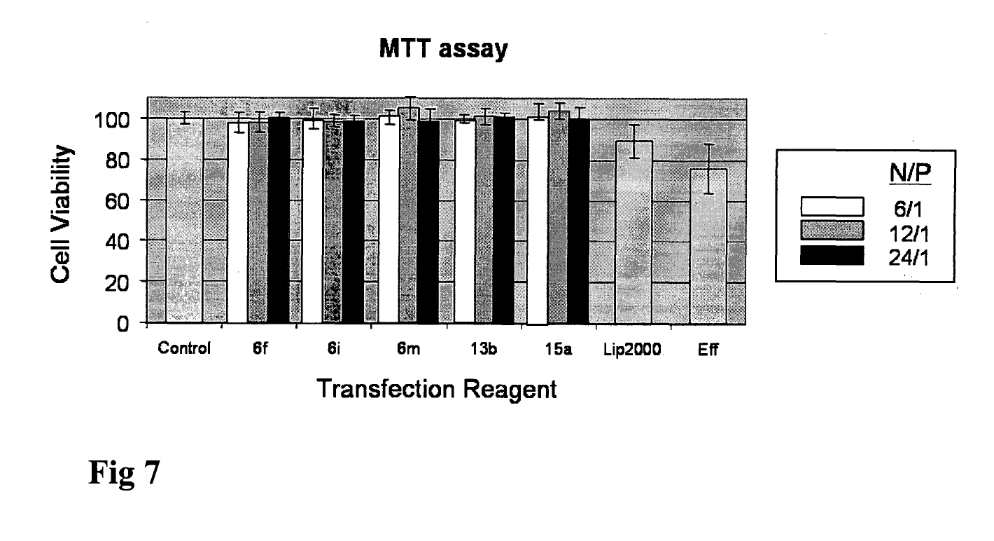

The results indicated that none of the compounds were toxic at the concentration used for transfection (N/P ratios 6/1, 12/1 and 24/1) The comparative data obtained with lipids 6f, 6i, 6m, 13b, and 15a of the invention, Lipofectamine™ 2000 and Effectene® Transfection Reagent is shown in FIG. 7.

Transfection of siRNA into GFP-Expressing Mouse Stem Cells

mES cells were grown in GMEM supplemented with 4 mM glutamine and 10% FBS in the absence of antibiotic until 80% confluence. Cells were then suspended using trypsin/EDTA and counted. 2×10⁴ cells in 150 μL of GMEM-CM per well were seeded in 96-well plates and incubated overnight. The day afterwards, the different lipoplex formulations were added. Each experiment was performed in quadruplicate, using Lipofectamine™ 2000 (Invitrogen) as a positive control and untreated cells as a negative control.

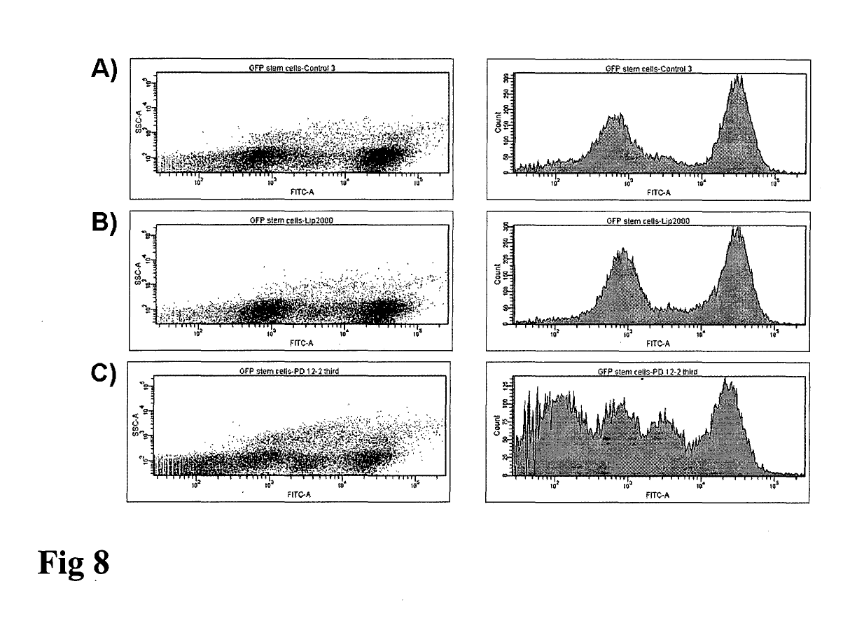

After incubation for two days at 37° C. and 5% CO₂ (change of media after transfection was not necessary), the green fluorescent protein (GFP) reduced expression was measured by flow cytometry. For the flow cytometry analysis, cells were washed twice with PBS, detached with trypsin/EDTA, harvested with 2% FBS in PBS, centrifuged and resuspended with 2% FBS in PBS and analyzed using a BD FACSaria flow cytometer. As shown in FIG. 8 preliminary assays using pentadecanoyl derivative 6f for transfecting siRNA resulted in 40% knock-down of GFP expression of GFP-transformed mouse stem cells, (better than Lipofectamine™ 2000). Thus FIG. 8 shows flow cytometry analysis of GFP-expressing mES cells after 48 h. using (A) an untransfected cell control; (B) Lipofectamine™ 2000; and (C) Pentadecanoyl derivative 6f, N/P 12, 1:2 mol mixture with DOPE.
In Vivo Transfection and Non-Invasive Luminescence Imaging from Living Mice

Mice were anesthetized and intubated following standard protocols. 16 μg of a luciferase-reporter plasmid (pLux, 4-6 mg/mL in PBS or another isotonic buffer) was complexed with Oleyl derivative 6i (N/P 12, 1:2 mol mixture with DOPE) following the protocol described before. Lipoplex formulation (total volume=50 μL) and naked plasmid (16 μg of plasmid in 50 μL of PBS) were then administrated into the lung by direct intratracheal instillation through peroral intubation. Mice were followed and analyzed on a daily basis, and all of them behaved healthily during the study.

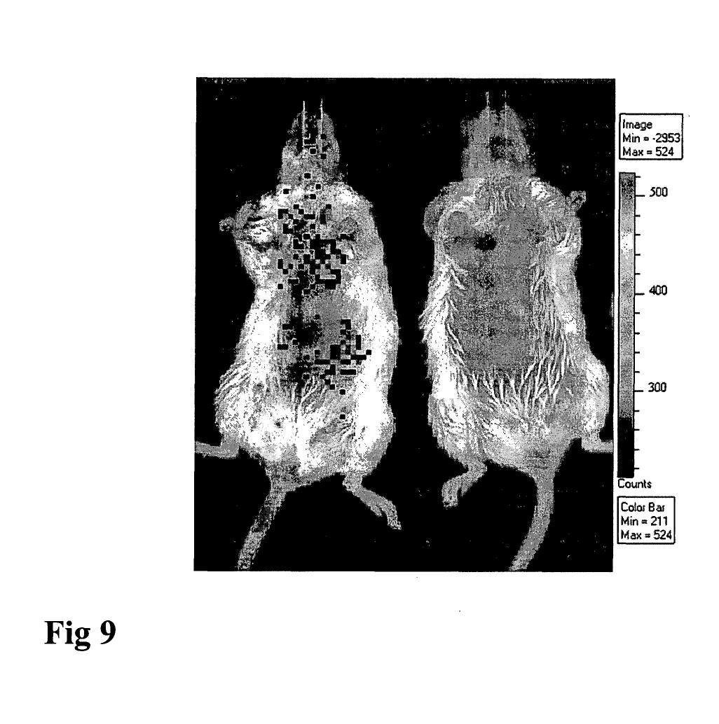

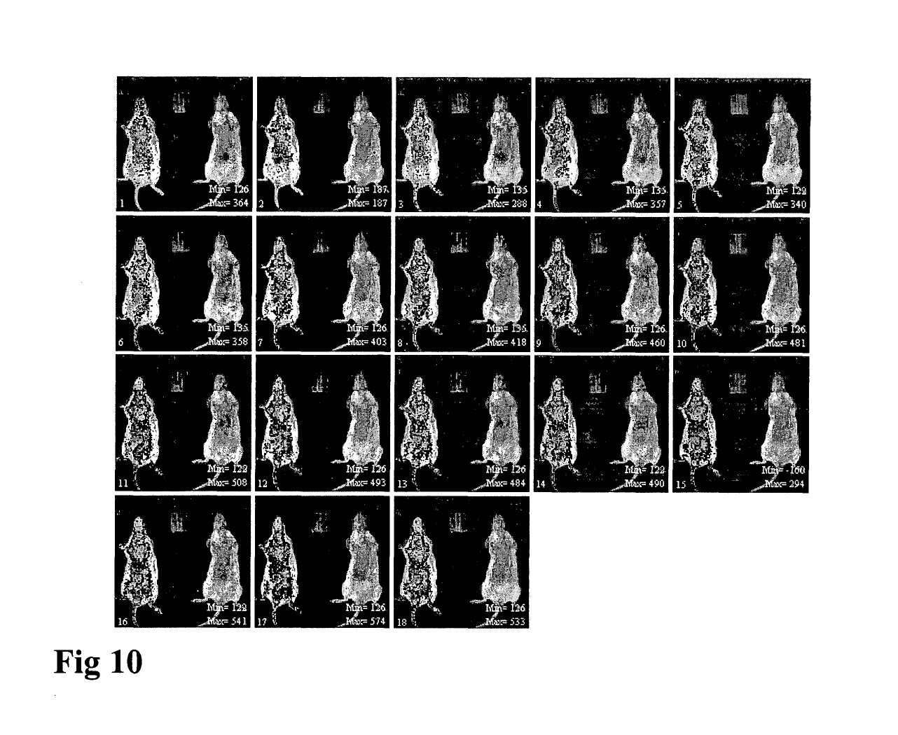

Firefly luciferin (15 mg/kg) was intraperitoneally administrated to the anesthetized mice half an hour before scanning for luminescence. Mice were then imaged every 2 minutes using an IVIS SPECTRUM imaging device. FIGS. 9 and 10 show the non-invasive in vivo luminescence imaging of anesthetized mice transfected with 16 μg of a luciferase-reporter plasmid (pLux) complexed with derivative 6i (left mouse) and naked plasmid (right mouse) 72 and 120 hours after instillation respectively. As shown in both FIGS. 9 and 10, positive luminescence was detected from the mouse transfected with derivative 6i (left mouse) and no luminescence detected from the mouse transfected with the naked plasmid (right mouse). Note that in the left mouse positive luminescence was detected not only from the lungs but also the mouth, guts (which is due to the partial spill over of instillation into the gastrointestinal track) and other body organs, which highlights the in vivo transfecting ability of derivative 6i.

1. A cationic lipid comprising a plurality of cationic moieties or cationic precursors within a plurality of headgroups, a lipophilic moiety and a linking moiety positioned between the lipophilic moiety and the headgroups, wherein the lipophilic moiety is connected to the linking moiety through a nitrogen-containing linkage and each of the headgroups is connected through an ester moiety to the linking moiety.

2. The cationic lipid of claim 1 wherein the plurality of headgroups are the same.

3. The cationic lipid of claim 1 wherein the plurality of cationic moieties or cationic precursors are the same.

4. The cationic lipid of claim 1 comprising two or three headgroups.

5. The cationic lipid of claim 1 wherein the linking moiety is based upon a compound having 3 hydroxyl groups and an amine group or 2 hydroxyl groups and an amine group.

6. The cationic lipid of claim 1 wherein the linking molecule is based upon 2-amino-2-hydroxymethyl-1,3-propanediol, or 2-amino-1,3-propanediol.

7. The cationic lipid of claim 1 wherein the headgroups comprise amine or guanidine groups.

8. The cationic lipid of claim 7 wherein the headgroups comprise primary, secondary, tertiary or quaternary amines.

9. The cationic lipid of claim 7 wherein the headgroup is an amine-containing acyl moiety.

10. The cationic lipid of claim 9 wherein the acyl moiety is derived from an amino acid.

11. The cationic lipid of claim 10 wherein the amino acid provides an α-aminoacyl, β-aminoacyl, γ-aminoacyl, δ-aminoacyl, or ε-aminoacyl moiety.

12. The cationic lipid of claim 10 wherein the aminoacyl moiety is glycinyl, β-alanyl, γ-aminobutyryl, 5-aminopentanoyl, 6-aminohexanoyl, lysinyl or arginyl.

13. The cationic lipid of claim 10 wherein the aminoacyl moiety is glycinyl, β-alanyl or γ-aminobutyryl.

14. The cationic lipid of claim 9 wherein the acyl moiety is derived from a guanidino-containing carboxylic acid.

15. The cationic lipid of claim 14 wherein the amino acid provides an α-guanidinoacyl, β-guanidinoacyl, γ-guanidinoacyl, δ-guanidinoacyl, or ε-guanidinoacyl moiety.

16. The cationic lipid of claim 14 wherein the guanidinoacyl moiety is guanidinoacetyl, β-guanidinopropionyl, γ-guanidinobutyryl, 5-guanidinopentanoyl, 6-guanidinohexanoyl.

17. The cationic lipid of claim 14 wherein the lipophilic moiety comprises a lipophilic motif of formula (I):
(D-E)ₙ-A- (I)
wherein:
n is 1 where A is absent or divalent and 2 where A is trivalent;
D is a saturated or unsaturated hydrocarbon moiety comprising from 1 to 100 carbon atoms, more usually 10 to 30 carbon atoms, for example 12 to 24 carbon atoms, which comprises one or more straight-chain, branched, or cyclic regions, which moiety may contain no or one or more heteroatoms selected from the group comprising oxygen, sulfur and nitrogen, and which moiety may be unsubstituted or substituted either internally or externally with one or more, e.g. one or two, functional groups selected from the group comprising hydroxyl, oxo, mercapto, thio, sulfoxy, sulfonyl, amino, carboxy, keto and ester;
E is absent or is selected from the group comprising carbonyl, amine, ether, thioether, phosphatidyl, sulfonyl, sulfoxide, ester, carbamoyl, carbamate and amide; and A is absent or is a di or trivalent of one of the following formulae:
-G-D-G-, (-D-G)₂-J-G-D-G-, -G-D(G-)-K-G-D-G-
or (-G-D)-D(G-)-K-E-D-G-D-G-,
in particular -G-D-G-, (-D-G)₂-J-G-D-G-, - or (-G-D)-D(G-)-K-E-D-G-D-G-,
wherein
each D and E is independently as hereinbefore defined;
each G is independently selected from the group comprising carbonyl, amine, ether, thioether, phosphatidyl, sulfonyl, sulfoxide, ester, carbamoyl, carbamate, amide and urea;
J is N or CH; and
K is NH or CH₂.

18. The cationic lipid of claim 17, wherein A is absent and E is present and is a carbonyl, amide or ester.

19. The cationic lipid of claim 18 wherein the nitrogen-containing linkage comprises E.

20. The cationic lipid of claim 17 wherein A is absent.

21. The cationic lipid of claim 17 wherein A is present, and is a moiety of formula -G-D-G-.

22. The cationic lipid of claim 17 wherein A is present, and is a moiety of formula (-D-G)₂-J-G-D-G-, for example wherein the (-D-G)₂-J- moiety is (—O—CH₂)₂—CH—O—.

23. The cationic lipid of claim 17 wherein A is present, and is a moiety of formula (-G-D)-D(G-)-K-E-D-G-D-G-.

24. The cationic lipid of claim 23 wherein (-G-D)-D(G-)-K-E-D- is of the formula (—OCH₂)—CH(O—)—CH₂—O—P(═O)(—O⁻)—O—(CH₂)₂—N(H)—.

25. The cationic lipid of claim 17 wherein A is present, and is a moiety of formula -G-D(G-)-K-G-D-G-.

26. The cationic lipid of claim 25 wherein -G-D(G-)-K- is of the formula —O—C(═O)—(CH₂)ᵣ—CH(C(═O)(—O—))—NH—, wherein r is from 1 to 10, for example 1 to 6.

27. The cationic lipid of claim 17 wherein each G may be carbonyl or amide.

28. The cationic lipid of claim 17 wherein D in -G-D-G- is an alkylene moiety of the formula —(CH₂)ᵣ— wherein r is from 1 to 10, e.g. 2 to 6.

29. The cationic lipid of claim 28 wherein D in -G-D-G- is derived from γ-aminobutyric acid or succinic acid.

30. The cationic lipid of claim 17 wherein D-E- comprises a saturated or unsaturated fatty alkyl chain.

31. The cationic lipid of claim 30 wherein D- is decyl, undecyl, dodecyl, tridecyl, tetradecyl, pentadecyl, hexadecyl, octadecyl, 7-octadecenyl, eicosyl or tetraeicosyl; or is represented as CH₃(CH₂)ₚ— wherein p is from 5 to 100, more usually 10 to 30, for example 12 to 24.

32. The cationic lipid of claim 17 wherein D-E- is a saturated or unsaturated fatty acyl chain.

33. The cationic lipid of claim 32 wherein D-E- is decanoyl, undecanoyl, dodecanoyl, tridecanoyl, tetradecanoyl, pentadecanoyl, hexadecanoyl, octadecanoyl, 7-octadecenoyl, eicosanoyl or tetraeicosanoyl; or is represented as CH₃(CH₂)_{q}C(═O)— wherein q is from 5 to 100, more usually 10 to 30, for example 12 to 24.

34. The cationic lipid of claim 17 wherein D-E- is a saturated or unsaturated fatty alkyloxy chain.

35. The cationic lipid of claim 34 wherein D-E- is decyloxy, undecyloxy, dodecyloxy, tridecyloxy, tetradecyloxy, pentadecyloxy, hexadecyloxy, octadecyloxy, 9-octadecenyloxy (oleyloxy), eicosyloxy, or tetraeicosyloxy; or is represented as CH₃(CH₂)_{q}C—O— wherein q is from 5 to 100, more usually 10 to 30, for example 12 to 24.

36. The cationic lipid of claim 20 wherein E is absent.

37. The cationic lipid of claim 17 wherein the D-E- moiety comprises cholesteryloxycarbonyl.

38. The cationic lipid of claim 1 wherein the nitrogen linkage is an amide, urea or carbamate linkage.

39. The cationic lipid of claim 1 wherein the nitrogen-containing linkage is an amide linkage.

40. The cationic lipid of claim 1 wherein the cationic lipid comprises cationic moieties and is associated with anions selected from the group comprising halide anions such as fluoro, iodo, bromo and chloro, acetate, trifluoroacetate, bisulfate or methyl sulfate.

41. A composition comprising a cationic lipid according to claim 1 in combination with an additional lipid.

42. The composition of claim 41 wherein the additional lipid is a neutral lipid selected from the group comprising diolelyl phosphatidyl ethanolamine, 1,2-dioleyl-glycero-3-phosphocholine and cholesterol.

43. The composition of claim 41 wherein the additional lipid is present in molar ratio, of additional lipid:cationic lipid, of about 1:10 to 10:1, more usually from about 1:5 to 5:1, e.g. about 3:1 to 1:1.

44. A composition comprising a cationic lipid according to claim 1 in combination with a biologically active molecule, such as a polynucleotide molecule.

45. The composition of claim 44 which is an aqueous emulsion.

46. The composition of claim 45 comprising a culture medium.

47. The composition of claim 44 further comprising an additional lipid as defined in any one of claims 41 to 43.

48. The composition of claim 44 wherein the polynucleotide is a DNA or an RNA, e.g. siRNA.

49. The composition of claim 44 wherein the ratio of positive charges provided by the cationic lipid to negative charges provided by the polynucleotide is about 2 to 100:1, e.g. about 2 to 50:1, typically between about 5:1 and 25:1.

50. A method for transfecting a polynucleotide into a cell comprising contacting a cell with a composition according to claim 44.

51. The method of claim 50 which is an in vitro or in vivo method of transfecting cells, particularly eukaryotic cells or tissue such as animal cells or tissue, and in particular mammalian cells or tissue, in particular human cells or tissue.

52. Use of a composition according to claim 44 in the manufacture of a medicament.

53. A kit of parts comprising one or more of the cationic lipid of claim 1, or a composition comprising such a cationic lipid in combination with an additional lipid, and at least one additional component selected from the group consisting of a cell, cells, a cell culture media, a nucleic acid, a transfection enhancer and instructions for transfecting a cell or cells.
